(19) United States
(12) Patent Application Publication
(10) Pub. No.: US 2026/0035420 A1
(43) Pub. Date: Feb. 5, 2026

(54) AGGREGATION-RESISTANT VARIANTS OF TDP-43

(71) Applicant: Regeneron Pharmaceuticals, Inc., Tarrytown, NY (US)

(72) Inventors: Aarti Sharma-Kanning, Croton-on-Hudson, NY (US); Sarah Tisdale, Stamford, CT (US); David Frendewey, New York, NY (US)

(21) Appl. No.: 19/100,696

(22) PCT Filed: Aug. 4, 2023
(86) PCT No.: PCT/US2023/071677

§ 371 Date: Feb. 3, 2025

Related U.S. Application Data
(60) Provisional application No. 63/370,527, filed on Aug. 5, 2022.

(51) Int. Cl. C07K 14/47; A01K 67/0275; A61K 38/00; A61P 25/28; C12N 5/071; C12N 9/22; C12N 15/113; C12N 15/864

(52) U.S. Cl. CPC C07K 14/4702; A01K 67/0275; A61K 38/00; A61P 25/28; C12N 5/0602; C12N 9/226; C12N 15/1135; C12N 15/8645; A01K 2227/105; C07K 2319/80; C12N 2310/11; C12N 2310/20; C12N 2740/15043; C12N 2750/14143

(57) ABSTRACT

Provided herein are TDP-43 variants in which a prion-like domain (PLD) of the TDP-43 variant is mutated to have more aromatic amino acids and/or aromatic amino acids that are more evenly spaced than in a PLD from a wild type TDP-43, nucleic acids encoding such TDP-43 variants, and methods of using such TDP-43 variants, for example, methods of treating TDP-43 proteinopathies.

### CROSS-REFERENCE TO RELATED APPLICATIONS

This application claims the benefit of U.S. Application No. 63/370,527, filed Aug. 5, 2022, which is herein incorporated by reference in its entirety for all purposes.

### REFERENCE TO A SEQUENCE LISTING SUBMITTED AS A TEXT FILE VIA EFS WEB

The Sequence Listing written in file 599567SEQLIST.xml is 122 kilobytes, was created on Aug. 4, 2023, and is hereby incorporated by reference.

### BACKGROUND

TAR DNA-binding protein 43 (TDP-43) is a ubiquitous protein encoded by the highly conserved TARDBP gene. TDP-43 is a predominantly nuclear RNA-binding protein similar to members of the heterogeneous nuclear ribonucleoprotein (hnRNP) family that is developmentally regulated and indispensable for early embryonic development. TDP-43 binds to thousands of RNAs and has a strong preference for UG rich sequences. TDP-43 auto-regulates its synthesis by promoting an alternative spicing event at the 3′ end of the pre-mRNA. Although the entire 3D structure of TDP-43 has yet to be resolved, several structural features of the TDP-43 protein have been identified. These include a nuclear localization signal (NLS), two RNA recognition motifs (RRM1 and RRM2), a putative nuclear export signal (NES), and a large domain in the carboxyl-terminal half of the protein that has been described as a low complexity, poorly ordered, or prion-like domain (PLD). TDP-43 has also been shown to be a disease signature protein associated with several neurodegenerative diseases including amyotrophic lateral sclerosis (ALS), in which 97% of ALS cases show a post-mortem pathology of cytoplasmic TDP-43 aggregates. These aggregates are ubiquitinated, hyperphosphorylated, and truncated. Additionally, mutations in TDP-43 are associated with ALS. Of these rare TDP-43 mutations, the majority are found in its prion-like domain. The correlation between the physiological functions of TDP-43 with these diseases remains unknown. Therefore, understanding the normal physiologic role of TDP-43 is essential.

The mechanisms by which RNA-binding proteins such as TDP-43 trigger neurodegeneration are not fully understood. TDP-43 is ubiquitously expressed and thought to be involved in multiple levels of RNA metabolism, including transcription, splicing, transport, and translation. Despite the critical roles that TDP-43 plays in maintaining cellular life, the structural and functional domains through which these functions are maintained are poorly defined.

### SUMMARY

Provided herein are TDP-43 variants in which a prion-like domain (PLD) of the TDP-43 variant is mutated to have more aromatic amino acids and/or aromatic amino acids that are more evenly spaced than in a PLD from a wild type TDP-43, nucleic acids encoding such TDP-43 variants, cells and animals comprising such variants or nucleic acids, methods of making such cells and animals, and methods of using such TDP-43 variants such as, for example, methods of treating TDP-43 proteinopathies such as amyotrophic lateral sclerosis (ALS).

In one aspect, provided is a TAR DNA-binding protein 43 (TDP-43) variant in which a prion-like domain (PLD) of the TDP-43 variant is mutated to have more aromatic amino acids and/or aromatic amino acids that are more evenly spaced than in a PLD from a wild type TDP-43. In some such TDP-43 variants, the PLD is mutated to have more aromatic amino acids. In some such TDP-43 variants, the PLD is mutated to have aromatic amino acids that are more evenly spaced than in the PLD from the wild type TDP-43. In some such TDP-43 variants, the PLD is mutated to have more aromatic amino acids and aromatic amino acids that are more evenly spaced than in the PLD from the wild type TDP-43.

In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 22. In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 63. In some such TDP-43 variants, the PLD in the TDP-43 variant comprises, consists essentially of, or consists of the sequence set forth in SEQ ID NO: 22. In some such TDP-43 variants, the PLD in the TDP-43 variant comprises, consists essentially of, or consists of the sequence set forth in SEQ ID NO: 63. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 23. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 64. In some such TDP-43 variants, the TDP-43 variant comprises, consists essentially of, or consists of the sequence set forth in SEQ ID NO: 23. In some such TDP-43 variants, the TDP-43 variant comprises, consists essentially of, or consists of the sequence set forth in SEQ ID NO: 64.

In some such TDP-43 variants, a portion of the PLD from the wild type TDP-43 is replaced with at least a portion of a PLD from a different RNA-binding protein in the variant TDP-43. In some such TDP-43 variants, the portion of the PLD from the wild type TDP-43 that is replaced is at least about 10, at least about 15, at least about 20, at least about 25, or at least about 28 amino acids. In some such TDP-43 variants, the portion of the PLD from the wild type TDP-43 that is replaced is between about 10 and about 50, between about 20 and about 40, or between about 25 and about 35 amino acids. In some such TDP-43 variants, the portion of the PLD that is replaced is about 28 amino acids. In some such TDP-43 variants, the portion of the PLD that is replaced comprises, consists essentially of, or consists of SEQ ID NO: 6, or the portion of the PLD that is replaced comprises, consists essentially of, or consists of SEQ ID NO: 47. In some such TDP-43 variants, the different RNA-binding protein is hnRNPA2B1 (e.g., human hnRNPA2B1 or mouse hnRNPA2B1). In some such TDP-43 variants, the portion of the PLD from hnRNPA2B1 is at least about 90%, at least about 95%, at least about 96%, at least about 97%, at least about 98%, at least about 99%, or 100% identical to the sequence set forth in SEQ ID NO: 24. In some such TDP-43 variants, the portion of the PLD from hnRNPA2B1 is at least about 90%, at least about 95%, at least about 96%, at least about 97%, at least about 98%, at least about 99%, or 100% identical to the sequence set forth in SEQ ID NO: 65. In some such TDP-43 variants, the portion of the PLD from hnRNPA2B1 comprises, consists essentially of, or consists of the sequence set forth in SEQ ID NO: 24. In some such TDP-43 variants, the portion of the PLD from hnRNPA2B1 comprises, consists essentially of, or consists of the sequence set forth in SEQ ID NO: 65. In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 25. In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 66. In some such TDP-43 variants, the PLD in the TDP-43 variant comprises, consists essentially of, or consists of the sequence set forth in SEQ ID NO: 25. In some such TDP-43 variants, the PLD in the TDP-43 variant comprises, consists essentially of, or consists of the sequence set forth in SEQ ID NO: 66. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 26. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 67. In some such TDP-43 variants, the TDP-43 variant comprises, consists essentially of, or consists of the sequence set forth in SEQ ID NO: 26. In some such TDP-43 variants, the TDP-43 variant comprises, consists essentially of, or consists of the sequence set forth in SEQ ID NO: 67.

In some such TDP-43 variants, the PLD from the wild type TDP-43 is replaced with a PLD from a different RNA-binding protein in the variant TDP-43. In some such TDP-43 variants, the different RNA-binding protein is hnRNPA2B1 (e.g., human hnRNPA2B1 or mouse hnRNPA2B1). In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 27 or 13. In some such TDP-43 variants, the PLD in the TDP-43 variant is at least about 90%, at least about 95%, at least about 96%, at least about 97%, at least about 98%, at least about 99%, or 100% identical to the sequence set forth in SEQ ID NO: 68 or 58. In some such TDP-43 variants, the PLD in the TDP-43 variant comprises, consists essentially of, or consists of the sequence set forth in SEQ ID NO: 27 or 13. In some such TDP-43 variants, the PLD in the TDP-43 variant comprises, consists essentially of, or consists of the sequence set forth in SEQ ID NO: 68 or 58. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 28. In some such TDP-43 variants, the TDP-43 variant is at least about 90%, at least about 95%, at least about 96%, at least about 97%, at least about 98%, at least about 99%, or 100% identical to the sequence set forth in SEQ ID NO: 69. In some such TDP-43 variants, the TDP-43 variant comprises, consists essentially of, or consists of the sequence set forth in SEQ ID NO: 28. In some such TDP-43 variants, the TDP-43 variant comprises, consists essentially of, or consists of the sequence set forth in SEQ ID NO: 69.

In some such TDP-43 variants, the TDP-43 variant is less prone to aggregation than the wild type TDP-43. In some such TDP-43 variants, the TDP-43 variant retains functions of wild type TDP-43 in splicing regulation. In some such TDP-43 variants, the TDP-43 variant is predominantly nuclear and/or retains the subcellular distribution of wild type TDP-43. In some such TDP-43 variants, the TDP-43 variant retains functions of wild type TDP-43 during embryonic development. In some such TDP-43 variants, the TDP-43 variant is a human TDP-43 variant.

Some such TDP-43 variants are for use in the treatment of a TDP-43 proteinopathy in a subject, optionally wherein the TDP-43 proteinopathy is amyotrophic lateral sclerosis (ALS). Some such TDP-43 variants are for use in the prevention of a TDP-43 proteinopathy in a subject, optionally wherein the TDP-43 proteinopathy is amyotrophic lateral sclerosis (ALS). Some such TDP-43 variants are for the manufacture of a medicament for the treatment of a TDP-43 proteinopathy, optionally wherein the TDP-43 proteinopathy is amyotrophic lateral sclerosis (ALS). In another aspect, provided is use of any of the above TDP-43 variants for the manufacture of a medicament for the prevention of a TDP-43 proteinopathy, optionally wherein the TDP-43 proteinopathy is amyotrophic lateral sclerosis (ALS).

In another aspect, provided are nucleic acids encoding any of the above TDP-43 variants. In some such nucleic acids, the nucleic acid is a messenger RNA. In some such nucleic acids, the nucleic acid comprises DNA, optionally wherein the DNA comprises a complementary DNA (cDNA). In some such nucleic acids, the nucleic acid is in an expression construct comprising a promoter operably linked to the nucleic acid encoding the TDP-43 variant, optionally wherein the promoter is a neuron-specific promoter or a constitutive promoter. In some such nucleic acids, the promoter is a constitutive promoter, a tissue-specific promoter, or an inducible promoter. In some such nucleic acids, the promoter is a neuron-specific promoter, optionally wherein the promoter is a synapsin-1 promoter, and optionally wherein the promoter is a human synapsin-1 promoter.

In some such nucleic acids, the nucleic acid is in a vector. In some such nucleic acids, the vector is a viral vector. In some such nucleic acids, the viral vector is a lentivirus vector or an adeno-associated virus (AAV) vector. In some such nucleic acids, the vector is the AAV vector, optionally wherein the AAV vector is an AAV-PHP.eB vector.

In some such nucleic acids, the nucleic acid is codon-optimized for expression in human cells.

In another aspect, provided are cells comprising any of the above TDP-43 variants or any of the above nucleic acids encoding TDP-43 variants. In some such cells, the TDP-43 variant is expressed. In some such cells, the cell is a mammalian cell. In some such cells, the mammalian cell is a human cell, a rodent cell, a mouse cell, or a rat cell. In some such cells, the cell is the human cell. In some such cells, the cell is a neuron, a glial cell, or a muscle cell. In some such cells, the cell is in vivo in a subject. In some such cells, the cell is a neuron in the brain of the subject.

In some such cells, endogenous TDP-43 is not expressed in the cell. In some such cells, the endogenous TARDBP genomic locus comprises a mutation that prevents expression of endogenous TDP-43 in the cell. In some such cells, the cell further comprises an agent that reduces or eliminates expression of endogenous TDP-43 in the cell. In some such cells, the agent comprises an antisense oligonucleotide or an RNAi agent targeting endogenous TARDBP messenger RNA or a nucleic acid encoding the antisense oligonucleotide or the RNAi agent. In some such cells, the agent comprises a nuclease agent targeting the endogenous TARDBP genomic locus or one or more nucleic acids encoding the nuclease agent. In some such cells, the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In some such cells, the nuclease agent is the Cas protein and the guide RNA, optionally wherein the Cas protein is a Cas9 protein.

In some such cells, the cell has a genetically modified endogenous TARDBP genomic locus, wherein the nucleic acid is integrated at the endogenous TARDBP genomic locus. In some such cells, the cell is heterozygous for the integrated nucleic acid. In some such cells, the cell is homozygous for the integrated nucleic acid. In some such cells, the nucleic acid is operably linked to the endogenous TARDBP promoter. In some such cells, the TDP-43 variant is expressed from the endogenous TARDBP genomic locus. In some such cells, the TDP-43 variant replaces expression of the endogenous TDP-43.

In some such cells, the cell has reduced TDP-43 aggregation compared to a control cell without the TDP-43 variant or the nucleic acid.

In another aspect, provided are non-human animals comprising any of the above TDP-43 variants or any of the above nucleic acids encoding TDP-43 variants. In some such non-human animals, the TDP-43 variant is expressed. In some such non-human animals, the non-human animal is a mammal. In some such non-human animals, the non-human animal is a rodent, a mouse, or a rat. In some such non-human animals, the non-human animal is the mouse. In some such non-human animals, the TDP-43 variant or the nucleic acid is in a neuron, a glial cell, or a muscle cell in the non-human animal. In some such non-human animals, the TDP-43 variant or the nucleic acid is in the neuron. In some such non-human animals, the neuron is in the brain of the non-human animal.

In some such non-human animals, endogenous TDP-43 is not expressed in the non-human animal. In some such non-human animals, the endogenous TARDBP genomic locus comprises a mutation that prevents expression of endogenous TDP-43 in the non-human animal. In some such non-human animals, the non-human animal comprises an agent that reduces or eliminates expression of endogenous TDP-43 in the non-human animal. In some such non-human animals, the agent comprises an antisense oligonucleotide or an RNAi agent targeting endogenous TARDBP messenger RNA or a nucleic acid encoding the antisense oligonucleotide or the RNAi agent. In some such non-human animals, the agent comprises a nuclease agent targeting the endogenous TARDBP genomic locus or one or more nucleic acids encoding the nuclease agent. In some such non-human animals, the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In some such non-human animals, the nuclease agent is the Cas protein and the guide RNA, optionally wherein the Cas protein is a Cas9 protein.

In some such non-human animals, the non-human animal has a genetically modified endogenous TARDBP genomic locus, wherein the nucleic acid is integrated at the endogenous TARDBP genomic locus. In some such non-human animals, the non-human animal is heterozygous for the integrated nucleic acid. In some such non-human animals, the non-human animal is homozygous for the integrated nucleic acid. In some such non-human animals, the nucleic acid is operably linked to the endogenous TARDBP promoter. In some such non-human animals, the TDP-43 variant is expressed from the endogenous TARDBP genomic locus. In some such non-human animals, the TDP-43 variant replaces expression of the endogenous TDP-43.

In some such non-human animals, the non-human animal has reduced TDP-43 aggregation compared to a control non-human animal without the TDP-43 variant or the nucleic acid.

In another aspect, provided are methods of making any of the above non-human animals, comprising administering the TDP-43 variant or the nucleic acid to the non-human animal. In another aspect, provided are methods of making any of the above non-human animals, comprising: (I) (a) modifying the genome of a pluripotent non-human animal cell to comprise the genetically modified endogenous TARDBP genomic locus; (b) identifying or selecting the genetically modified pluripotent non-human animal cell comprising the genetically modified endogenous TARDBP genomic locus; (c) introducing the genetically modified pluripotent non-human animal cell into a non-human animal host embryo; and (d) gestating the non-human animal host embryo in a surrogate mother; or (II) (a) modifying the genome of a non-human animal one-cell stage embryo to comprise the genetically modified endogenous TARDBP genomic locus; (b) selecting the genetically modified non-human animal one-cell stage embryo comprising the genetically modified endogenous TARDBP genomic locus; and (c) gestating the genetically modified non-human animal one-cell stage embryo in a surrogate mother.

In another aspect, provided are methods comprising administering to a cell: (i) any of the above TDP-43 variants; or (ii) any of the above nucleic acids encoding a TDP-43 variant. In some such methods, the TDP-43 variant is expressed. In some such methods, the cell is a mammalian cell. In some such methods, the mammalian cell is a human cell, a rodent cell, a mouse cell, or a rat cell. In some such methods, the cell is the human cell. In some such methods, the cell is a neuron, a glial cell, or a muscle cell. In some such methods, the cell is in vivo in a subject. In some such methods, the cell is a neuron in the brain of the subject. In some such methods, the TDP-43 variant or the nucleic acid is administered to the subject via intracerebroventricular injection, intracranial injection, or intrathecal injection.

In some such methods, the method comprises administering to the cell an agent that reduces or eliminates expression of endogenous TDP-43 in the cell. In some such methods, the agent comprises an antisense oligonucleotide or an RNAi agent targeting endogenous TARDBP messenger RNA or a nucleic acid encoding the antisense oligonucleotide or the RNAi agent. In some such methods, the agent comprises a nuclease agent targeting the endogenous TARDBP genomic locus or one or more nucleic acids encoding the nuclease agent. In some such methods, the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In some such methods, the nuclease agent is the Cas protein and the guide RNA, optionally wherein the Cas protein is a Cas9 protein.

In some such methods, the method comprises administering the nucleic acid encoding the TDP-43 variant, wherein the nuclease agent cleaves the endogenous TARDBP genomic locus, the nucleic acid encoding the TDP-43 variant is inserted at or recombines with the cleaved endogenous TARDBP genomic locus, wherein the TDP-43 variant is expressed from the endogenous TARDBP genomic locus. In some such methods, the TDP-43 variant replaces expression of the endogenous TDP-43.

In some such methods, endogenous TDP-43 in the cell is prone to aggregation, and wherein the method reduces TDP-43 aggregation in the cell. In some such methods, there is aberrant splicing regulation by endogenous TDP-43 in the cell, and wherein the TDP-43 variant rescues aberrant TDP-43 splicing regulation in the cell. In some such methods, there is aberrant subcellular distribution of endogenous TDP-43 in the cell, and wherein the TDP-43 variant rescues aberrant subcellular distribution of endogenous TDP-43 in the cell.

In another aspect, provided are methods of treating a TDP-43 proteinopathy in a subject. Some such methods comprise administering to one or more cells in the subject: (i) any of the above TDP-43 variants; or (ii) any of the above nucleic acids encoding a TDP-43 variant. In some such methods, the TDP-43 variant is expressed in the one or more cells in the subject. In another aspect, provided are methods of preventing a TDP-43 proteinopathy in a subject. Some such methods comprise administering to one or more cells in the subject: (i) any of the above TDP-43 variants; or (ii) any of the above nucleic acids encoding a TDP-43 variant. In some such methods, the TDP-43 variant is expressed in the one or more cells in the subject.

In some such methods, the TDP-43 proteinopathy is amyotrophic lateral sclerosis (ALS). In some such methods, the subject is a mammal. In some such methods, the subject is a human. In some such methods, the one or more cells comprise neurons in the brain of the subject. In some such methods, the TDP-43 variant or the nucleic acid is administered to the subject via intracerebroventricular injection, intracranial injection, or intrathecal injection.

In some such methods, the method further comprises administering to the one or more cells an agent that reduces or eliminates expression of endogenous TDP-43 in the one or more cells. In some such methods, the agent comprises an antisense oligonucleotide or an RNAi agent targeting endogenous TARDBP messenger RNA or a nucleic acid encoding the antisense oligonucleotide or the RNAi agent. In some such methods, the agent comprises a nuclease agent targeting the endogenous TARDBP genomic locus or one or more nucleic acids encoding the nuclease agent. In some such methods, the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In some such methods, the nuclease agent is the Cas protein and the guide RNA, optionally wherein the Cas protein is a Cas9 protein.

In some such methods, the method comprises administering the nucleic acid encoding the TDP-43 variant, wherein the nuclease agent cleaves the endogenous TARDBP genomic locus in the one or more cells, the nucleic acid encoding the TDP-43 variant is inserted at or recombines with the cleaved endogenous TARDBP genomic locus, wherein the TDP-43 variant is expressed from the endogenous TARDBP genomic locus and replaces expression of the endogenous TDP-43.

In some such methods, endogenous TDP-43 in the one or more cells is prone to aggregation, and wherein the method reduces TDP-43 aggregation in the one or more cells. In some such methods, there is aberrant splicing regulation by endogenous TDP-43 in the one or more cells, and wherein the TDP-43 variant rescues aberrant TDP-43 splicing regulation in the one or more cells. In some such methods, there is aberrant subcellular distribution of endogenous TDP-43 in the one or more cells, and wherein the TDP-43 variant rescues aberrant subcellular distribution of endogenous TDP-43 in the one or more cells.

### BRIEF DESCRIPTION OF THE FIGURES

FIG. 1 shows amino acid composition of the prion like domains (PLD) from TDP-43, hnRNPA1, and hnRNPA2B1. Aromatic residues are highlighted in orange. Biochemical studies have shown that the even spacing of aromatic residues throughout PLDs allows efficient liquid-liquid phase separation (LLPS) but prevents the irreversible associations that lead to aggregation. The PLD of TDP-43 has fewer and less evenly spaced aromatic amino acids than other RNA-binding proteins, suggesting that re-organizing the spacing of aromatic residues throughout the PLD could prevent aggregation.

FIG. 2A shows the structure of TDP-43. FIG. 2B shows that to determine if the PLD of another RNA binding protein could substitute for the PLD of TDP-43, we replaced the PLD of TDP-43 with a PLD from a less aggregation-prone RNA binding protein, hnRNPA2B1.

FIG. 3 shows that the wild type and PLDswap TDP-43 proteins were detected at the expected size of about 43 kDa. Using an antibody that recognizes the N-terminus of TDP-43, mutant TDP-43 polypeptide lacking a functional PLD redistributed from the nucleus to the cytoplasm in ES cell-derived motor neurons. Like wild type TDP-43, TDP-43 PLDswap is predominantly nuclear. As expected, an antibody that recognizes the C-terminus of a wild type TDP-43 protein was unable to detect the TDP-43 PLDswap protein (data not shown).

FIG. 4 shows that embryos lacking a functional TDP-43 protein (TDP-43^{-/-}) were not viable and did not survive beyond the E3.5 stage. Similarly, embryos expressing only a TDP-43 protein lacking a functional NLS (^{ΔNLS/-}) or only a TDP-43 protein lacking a functional PLD (TDP-43^{ΔPLD/-}) were not viable, although such embryos survived longer, suggesting that they can compensate for some of the embryonic functions of TDP-43. Embryos expressing only the PLDswap protein were able to survive until birth indicating that this form can fully compensate for wild type TDP-43 during embryogenesis.

FIG. 5 shows RT-PCR analysis of the indicated TDP-43-dependent splicing events. The specific splicing event being monitored is indicated in the mRNA schematics on the right, and the primer locations are indicated with the black arrows. Cryptic exons are indicated as red boxes in the schematics. In ES cell derived MNs where ΔNLS and ΔNES are the only form of the TDP-43 protein there is a clear loss of function in splicing regulation. In mutants where ΔPLD is the only form of TDP-43 this loss of function is not as severe. In ES-cell derived MNs where the only form of TDP-43 is the PLDswap chimeric protein, TDP-43 function is mostly restored. Adnp2 and Dnajc5 assays are monitoring the aberrant inclusion of a cryptic exon, while Poldip3 and Tsn are monitoring alternative exon skipping and Sortilin1 is monitoring alternative exon inclusion.

FIGS. 6A-6B show PLDswap neonatal survival. Mice harboring the PLDswap protein as the only form of TDP-43 following Cre-mediated removal of the WT allele either ubiquitously (CAG-Cre) or restricted to neurons (SYN-Cre) (FIG. 6A) survive at similar levels compared to WT heterozygous control mice (FIG. 6B). Survival plots of TDP-43^{ΔEx3/ΔEx3} (CAG-Cre, n=18; SYN-Cre, n=12), TDP-43^{ΔEx3/WT}(CAG-Cre, n=7; SYN-Cre, n=9), and TDP-43^{ΔEx3/PLDswap} (CAG-Cre, n=5; SYN-Cre, n=7) following Cre-mediated removal of the conditional WT allele across genotypes. Median survival times are as follows: for CAG-Cre: TDP-43^{ΔEx3}/ΔEx3: 4 weeks; TDP-43^{ΔEx3/WT}: 17.36 weeks; TDP-43^{ΔEx3/PLDswap}: 29.43 weeks; for SYN-Cre: TDP-43^{ΔEx3/ΔEx3}: 3.93 weeks; TDP-43^{ΔEx3/WT}: 52 weeks; TDP-43^{ΔEx3/PLDswap}: 37 weeks.

FIG. 7 shows PLDswap neonatal RT-PCR analysis of TDP-43-dependent splicing. RT-PCR analysis of the indicated TDP-43-dependent splicing events is shown. The specific splicing event being monitored is indicated in the mRNA schematics on the right, and the primer locations are indicated with the black arrows. Adnp2 is monitoring the aberrant inclusion of a cryptic exon, while Tsn is monitoring alternative exon skipping. The cryptic exon in Adnp2 is indicated as a box between exons 2 and 3 in the schematic. Loss of TDP-43 (ΔEx3/ΔEx3) triggers mis-splicing of both Adnp2 and Tsn, which both trend towards rescue when PLDswap is the only form of TDP-43.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous TARDBP sequence of an animal refers to a native TARDBP sequence that naturally occurs at the TARDBP locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a TAR DNA-binding protein 43 (TDP-43) protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values: 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are TDP-43 variants in which a prion-like domain (PLD) of the TDP-43 variant is mutated to have more aromatic amino acids and/or aromatic amino acids that are more evenly spaced than in a PLD from a wild type TDP-43, nucleic acids encoding such TDP-43 variants, and methods of using such TDP-43 variants such as, for example, methods of treating TDP-43 proteinopathies such as amyotrophic lateral sclerosis (ALS). ALS is a devastating neurodegenerative disease that affects motor neurons, paralysis and eventual death. A nearly universal pathological finding in postmortem examinations of ALS patient tissue is the accumulation of TDP-43 (transactive response DNA binding protein 43 kDa) in cytoplasmic inclusions. TDP-43 is a predominantly nuclear RNA binding protein similar in structure to members of the heterogeneous nuclear ribonucleoprotein (hnRNP) family that is required for the viability of all mammalian cells and the normal development and life of animals. The redistribution of TDP-43 from the nucleus to the cytoplasm and its accumulation in insoluble aggregates are two key diagnostic hallmarks of ALS disease. TDP-43's biological functions may have yet to be fully elucidated, but there is evidence that the protein participates in the regulation of pre-messenger RNA (pre-mRNA) splicing by preventing the use of cryptic exons in large introns and by influencing alternative splicing of several pre-mRNAs. TDP-43 is also proposed to have functions in the cytoplasm, perhaps in the shuttling of RNAs between the nucleus and cytoplasm and in the transport of mRNAs within the axons of neurons.

Several structural features of the TDP-43 protein have been identified, including a nuclear localization signal (NLS), two RNA recognition motifs (RRM1 and RRM2), a putative nuclear export signal (NES), and a large domain in the carboxyl-terminal half of the protein that has been described as a low complexity, poorly ordered, or prion-like domain (PLD). Of the mutations in TDP-43 that are associated with familial cases of ALS, most are found in the PLD.

A study of TDP-43 domain mutants identified two mutants (ΔPLD and ΔNLS) that were viable as ES-cell-derived motor neurons and caused TDP-43 protein to mislocalize from the nucleus to the cytoplasm, inducing cytoplasmic aggregation. Mice solely expressing these mutants (ΔPLD/− and ΔNLS/−) are embryonic lethal. Mice heterozygous for ΔNLS (ΔNLS/+) and ΔPLD (ΔPLD/+) are viable and show TDP-43 mislocalization, aggregation, insolubility, phosphorylation.

In ΔPLD/+ mice we can distinguish the mutant (˜30 kDa) protein from the native (˜43 kDa) WT protein. The TDP-43ΔPLD protein was found almost exclusively in the cytoplasmic fraction, but a some of the wild type TDP-43 was also found in the cytoplasmic fraction. The wild type protein but not the ΔPLD protein was phosphorylated and found in the detergent insoluble fraction. These results suggest that the presence of the mutant TDP-43 protein influences the behavior of the wild type protein. In other experiments we see that ΔNLS/+ mice have progressive degeneration of the motor system. Mice heterozygous for ΔPLD show degeneration but unlike the ΔNLS this degeneration does not progress between 4 and 6 months. Adult motor neurons can survive with TDP-43 that does not have a PLD, and we see fewer aggregates in cells that have ΔPLD as the only form of TDP-43. This suggests that the PLD of TDP-43 mediates aggregate formation, suggesting removal or reengineering the PLD of TDP-43 might provide a novel therapeutic approach. Biochemical studies have shown that the even spacing of aromatic residues throughout PLDs allows efficient liquid-liquid phase separation but prevents the irreversible associations that lead to aggregation. The PLD of TDP-43 has fewer and less evenly spaced aromatic amino acids than other RNA-binding proteins, suggesting that re-organizing the spacing of aromatic residues throughout the PLD could prevent aggregation.

To test this, we designed and created TDP-43 variants in which a prion-like domain (PLD) of the TDP-43 variant is mutated to have more aromatic amino acids and/or aromatic amino acids that are more evenly spaced than in a PLD from a wild type TDP-43. As one example, we created a PLDswap allele that replaces the PLD of TDP-43 with the PLD of hnRNPA2B1. The PLD of hnRNPA2B1 when compared to that of TDP-43 is comprised of evenly spaced aromatic residues. Embryonic stem (ES) cells and ES-cell-derived motor neurons with the PLDswap allele as the only form of TDP-43 are viable and display normal TDP-43 subcellular distribution. Replacement of TDP-43's PLD with the PLD of hNRNPA2B1 also rescues splicing defects associated with loss of functional TDP-43. This suggests a potential therapeutic modality could be to remove wild type TDP-43 and replace it with a modified, aggregation resistant form. It may also be that simply replacing the wild type protein would rescue phenotypes associated with the loss of functional TDP-43.

### II. TDP-43 Variants

Provided herein are variants of TAR DNA-binding protein 43 (TDP-43). Human TDP-43 is assigned UniProt reference number Q13148. The human gene encoding TDP-43 (TARDBP or TDP43) is assigned NCBI GeneID 23435 and is found at location 1p36.22 on chromosome 1 (assembly: GRCh38.p14 (GCF_000001405.40); location: NC_000001.11 (11012654 . . . 11030528)). At least two isoforms of human TDP-43 are known. The first isoform is 414 amino acids and is assigned UniProt reference number Q13148-1 and NCBI reference number NP_031401.1 (SEQ ID NO: 1). An exemplary coding sequence is assigned reference number CCDS122.1 (SEQ ID NO: 2), and an exemplary mRNA (cDNA) sequence is assigned reference number NM_007375.4 (SEQ ID NO: 3). The second isoform is 298 amino acids and is assigned UniProt reference number Q13148-2 (SEQ ID NO: 4).

Mouse TDP-43 is assigned UniProt reference number Q921F2. The mouse gene encoding TDP-43 (TARDBP or TDP43) is assigned NCBI GeneID 230908and is found at location 4; 4 E2 on chromosome 4 (assembly: GRCm39 (GCF_000001635.27); location: NC_000070.7 (148696839 . . . 148711672, complement)). The canonical isoform is 414 amino acids and is assigned UniProt reference number Q921F2-1 and NCBI reference number NP_663531.1 (SEQ ID NO: 43). An exemplary coding sequence is assigned reference number CCDS38971.1 (SEQ ID NO: 44), and an exemplary mRNA (cDNA) sequence is assigned reference number NM_145556.4 (SEQ ID NO: 45).

TDP-43 is a highly conserved and ubiquitously expressed RNA/DNA-binding protein belonging to the heterogeneous nuclear ribonucleoprotein (hnRNP) family. TDP-43 is pivotal in multiple cellular functions including regulation of RNA metabolism, mRNA transport, microRNA maturation and stress granule formation. In line with its nuclear and cytoplasmic functions, TDP-43 can shuttle between the nucleus and the cytoplasm, but under normal physiological conditions, localization is predominantly nuclear. Of relevance to brain function, TDP-43 appears to be critical for normal development of central neuronal cells in early stages of embryogenesis.

Dysfunction of TDP-43-related pathways has been increasingly recognized as an important pathogenic mechanism in neurodegenerative disease. Hyperphosphorylated and ubiquitinated TDP-43 cytoplasmic inclusions have been identified as a pathological feature of amyotrophic lateral sclerosis (ALS) and frontotemporal lobar disease (FTLD). Pathogenic missense mutations in the TARDBP gene, which encodes the TDP-43 protein, were subsequently identified as causative genetic mutations in both ALS and FTLD, although in a small percentage of familial cases. The vast majority of patients with ALS and FTLD do not harbor mutations in the TARDBP gene yet demonstrate widespread abnormalities involving TDP-43. TDP-43 deposition has been associated with an increasing number of neurodegenerative diseases, where it has been identified as the primary pathogenic factor, resulting in these disorders being designated as TDP-43 proteinopathies.

The protein structure of TDP-43 is comprised of an N-terminal region, nuclear localization signal (NLS), two RNA recognition motifs: RRM1 and RRM2, nuclear export signal (NES), and a C-terminal region encompassing a prion-like domain (PLD). PLDs are a subset of low complexity regions, enriched in uncharged polar amino acids and glycines, with similarities to the yeast prion protein that can be defined using a hidden Markov algorithm. PLDs are often found in RNA-binding proteins that drive protein aggregation in neurodegenerative disorders such as amyotrophic lateral sclerosis. Although predominantly localized in the nucleus, TDP-43 shuttles between the nucleus and the cytoplasm, a process mediated by active and passive transport, where it exerts physiological functions. In addition, TDP-43 localizes to the mitochondria where it associates with the mitochondrial genome and is important in the respiratory chain pathways. The best-known function of TDP-43 is in regulating the splicing of cryptic and alternative exons. Cryptic exons are intron sequences that are falsely recognized as exons by the splicing machinery. GU-rich TDP-43 binding sites are often found near cryptic exons in large introns. TDP-43 represses recognition of cryptic exons in large introns to promote normal splicing. Loss of TDP-43 results in aberrant splicing to cryptic exons, causing loss of normal RNA and protein. In addition, TDP-43 regulates the alternative splicing of specific target RNAs. This regulation includes both exon inclusion or exon exclusion, and can affect transcripts implicated in ALS pathogenesis.

The N-terminal domain is important in the formation of functional homodimers, which are critical for proper TDP-43 physiological function. Located within the N-terminal region is the NLS domain (amino acids 82-98) which mediates the import of TDP-43 into the nucleus where it exerts its physiological functions. The RNA-binding motifs (RRM1 (amino acids 106-176) and RRM2 (amino acids 191-262)) are essential for TDP-43 protein binding to RNA/DNA molecules, regulating transcription, translation, splicing and stability of mRNA, as well as mediating RNA export. In addition, TDP-43 forms ribonucleoprotein (RNP) granules that are important for the transportation of mRNA molecules and for promoting biogenesis of non-coding RNAs, such as microRNA (miRNA). Separately, the prion-like domain (PLD; amino acids 274-414; SEQ ID NO: 5 in human TDP-43; SEQ ID NO: 46 in mouse TDP-43) has been implicated in TDP-43 pathogenesis as this region regulates protein solubility and mediates pathological aggregation. TDP-43 is also important for the formation of stress granules, protecting the neuronal cells against cellular insults such as oxidative stress.

Biochemical studies have shown that the even spacing of aromatic residues throughout PLDs allows efficient liquid-liquid phase separation but prevents the irreversible associations that lead to aggregation. The PLD of TDP-43 has fewer and less evenly spaced aromatic amino acids than other RNA-binding proteins, suggesting that re-organizing the spacing of aromatic residues throughout the PLD could prevent aggregation. Provided herein are TDP-43 variants engineered to be less aggregation-prone than wild type TDP-43. Provided herein are TDP-43 variants (e.g., human TDP-43 variants) in which the PLD of the TDP-43 variant is mutated to have more aromatic amino acids and/or aromatic amino acids that are more evenly spaced than in a PLD from a wild type TDP-43. The three aromatic amino acids are tyrosine, phenylalanine, and tryptophan. In some cases, a TDP-43 variant described herein is mutated to have more aromatic amino acids than in a PLD from a wild type TDP-43. In some cases, a TDP-43 variant described herein is mutated to have aromatic amino acids that are more evenly spaced than in a PLD from a wild type TDP-43. In some cases, a TDP-43 variant described herein is mutated to have more aromatic amino acids and to have aromatic amino acids that are more evenly spaced than in a PLD from a wild type TDP-43.

In one example, the PLD of the TDP-43 variant is at least about 90%, at least about 95%, at least about 96%, at least about 97%, at least about 98%, at least about 99%, or 100% identical to the sequence set forth in SEQ ID NO: 22. In one example, the PLD of the TDP-43 variant comprises the sequence set forth in SEQ ID NO: 22. In one example, the PLD of the TDP-43 variant consists essentially of the sequence set forth in SEQ ID NO: 22. In one example, the PLD of the TDP-43 variant consists of the sequence set forth in SEQ ID NO: 22. In one example, the TDP-43 variant is at least about 90%, at least about 95%, at least about 96%, at least about 97%, at least about 98%, at least about 99%, or 100% identical to the sequence set forth in SEQ ID NO: 23. In one example, the TDP-43 variant comprises the sequence set forth in SEQ ID NO: 23. In one example, the TDP-43 variant consists essentially of the sequence set forth in SEQ ID NO: 23. In one example, the TDP-43 variant consists of the sequence set forth in SEQ ID NO: 23.

In one example, the PLD of the TDP-43 variant is at least about 90%, at least about 95%, at least about 96%, at least about 97%, at least about 98%, at least about 99%, or 100% identical to the sequence set forth in SEQ ID NO: 63. In one example, the PLD of the TDP-43 variant comprises the sequence set forth in SEQ ID NO: 63. In one example, the PLD of the TDP-43 variant consists essentially of the sequence set forth in SEQ ID NO: 63. In one example, the PLD of the TDP-43 variant consists of the sequence set forth in SEQ ID NO: 63. In one example, the TDP-43 variant is at least about 90%, at least about 95%, at least about 96%, at least about 97%, at least about 98%, at least about 99%, or 100% identical to the sequence set forth in SEQ ID NO: 64. In one example, the TDP-43 variant comprises the sequence set forth in SEQ ID NO: 64. In one example, the TDP-43 variant consists essentially of the sequence set forth in SEQ ID NO: 64. In one example, the TDP-43 variant consists of the sequence set forth in SEQ ID NO: 64.

In one example, a portion of the PLD from the wild type TDP-43 is replaced with at least a portion of a PLD from a different RNA-binding protein in the variant TDP-43. In one example, the portion of the PLD from the wild type TDP-43 that is replaced and/or the portion of a PLD from a different RNA-binding protein is at least about 10, at least about 15, at least about 20, at least about 25, or at least about 28 amino acids. In one example, the portion of the PLD from the wild type TDP-43 that is replaced and/or the portion of a PLD from a different RNA-binding protein is between about 10 and about 50, between about 20 and about 40, between about 25 and about 35, between about 10 and about 45, between about 10 and about 40, between about 10 and about 35, between about 10 and about 30, between about 15 and about 50, between about 20 and about 50, between about 25 and about 50, or between about 28 and about 50 amino acids. In another example, the portion of the PLD from the wild type TDP-43 that is replaced and/or the portion of a PLD from a different RNA-binding protein is between about 10 and about 140, between about 20 and about 140, between about 30 and about 140, between about 40 and about 140, between about 50 and about 140, between about 60 and about 140, between about 70 and about 140, between about 80 and about 140, between about 90 and about 140, between about 100 and about 140, between about 110 and about 140, between about 120 and about 140, between about 130 and about 140, between about 28 and about 130, between about 28 and about 120, between about 28 and about 110, between about 28 and about 100, between about 28 and about 90, between about 28 and about 80, between about 28 and about 70, between about 28 and about 60, between about 28 and about 50, or between about 28 and about 40 amino acids. In another example, the portion of the PLD from the wild type TDP-43 that is replaced and/or the portion of a PLD from a different RNA-binding protein is between about 10 and about 50, between about 20 and about 40, or between about 25 and about 35 amino acids. In another example, the portion of the PLD from the wild type TDP-43 that is replaced and/or the portion of a PLD from a different RNA-binding protein is about 28 amino acids.

For example, a highly conserved twenty-eight amino acid stretch within the PLD of TDP-43, shown to be important for aggregation in yeast, can be replaced with a sequence from a PLD from different RNA-binding protein. For example, the portion of the PLD from the wild type TDP-43 that is replaced can comprise the sequence set forth in SEQ ID NO: 6. In another example, the portion of the PLD from the wild type TDP-43 that is replaced consists essentially of the sequence set forth in SEQ ID NO: 6. In another example, the portion of the PLD from the wild type TDP-43 that is replaced consists of the sequence set forth in SEQ ID NO: 6. For example, the portion of the PLD from the wild type TDP-43 that is replaced can comprise the sequence set forth in SEQ ID NO: 47. In another example, the portion of the PLD from the wild type TDP-43 that is replaced consists essentially of the sequence set forth in SEQ ID NO: 47. In another example, the portion of the PLD from the wild type TDP-43 that is replaced consists of the sequence set forth in SEQ ID NO: 47.

In another example, the portion of a PLD from a different RNA-binding protein is at least about 90%, at least about 95%, at least about 96%, at least about 97%, at least about 98%, at least about 99%, or 100% identical to the sequence set forth in SEQ ID NO: 24. In another example, the portion of a PLD from a different RNA-binding protein comprises the sequence set forth in SEQ ID NO: 24. In another example, the portion of a PLD from a different RNA-binding protein consists essentially of the sequence set forth in SEQ ID NO: 24. In another example, the portion of a PLD from a different RNA-binding protein consists of the sequence set forth in SEQ ID NO: 24. In one example, the PLD of the TDP-43 variant is at least about 90%, at least about 95%, at least about 96%, at least about 97%, at least about 98%, at least about 99%, or 100% identical to the sequence set forth in SEQ ID NO: 25. In one example, the PLD of the TDP-43 variant comprises the sequence set forth in SEQ ID NO: 25. In one example, the PLD of the TDP-43 variant consists essentially of the sequence set forth in SEQ ID NO: 25. In one example, the PLD of the TDP-43 variant consists of the sequence set forth in SEQ ID NO: 25. In one example, the TDP-43 variant is at least about 90%, at least about 95%, at least about 96%, at least about 97%, at least about 98%, at least about 99%, or 100% identical to the sequence set forth in SEQ ID NO: 26. In one example, the TDP-43 variant comprises the sequence set forth in SEQ ID NO: 26. In one example, the TDP-43 variant consists essentially of the sequence set forth in SEQ ID NO: 26. In one example, the TDP-43 variant consists of the sequence set forth in SEQ ID NO: 26.

In another example, the portion of a PLD from a different RNA-binding protein is at least about 90%, at least about 95%, at least about 96%, at least about 97%, at least about 98%, at least about 99%, or 100% identical to the sequence set forth in SEQ ID NO: 65. In another example, the portion of a PLD from a different RNA-binding protein comprises the sequence set forth in SEQ ID NO: 65. In another example, the portion of a PLD from a different RNA-binding protein consists essentially of the sequence set forth in SEQ ID NO: 65. In another example, the portion of a PLD from a different RNA-binding protein consists of the sequence set forth in SEQ ID NO: 65. In one example, the PLD of the TDP-43 variant is at least about 90%, at least about 95%, at least about 96%, at least about 97%, at least about 98%, at least about 99%, or 100% identical to the sequence set forth in SEQ ID NO: 66. In one example, the PLD of the TDP-43 variant comprises the sequence set forth in SEQ ID NO: 66. In one example, the PLD of the TDP-43 variant consists essentially of the sequence set forth in SEQ ID NO: 66. In one example, the PLD of the TDP-43 variant consists of the sequence set forth in SEQ ID NO: 66. In one example, the TDP-43 variant is at least about 90%, at least about 95%, at least about 96%, at least about 97%, at least about 98%, at least about 99%, or 100% identical to the sequence set forth in SEQ ID NO: 67. In one example, the TDP-43 variant comprises the sequence set forth in SEQ ID NO: 67. In one example, the TDP-43 variant consists essentially of the sequence set forth in SEQ ID NO: 67. In one example, the TDP-43 variant consists of the sequence set forth in SEQ ID NO: 67.

In one example, the PLD from the wild type TDP-43 is replaced with a PLD from a different RNA-binding protein in the variant TDP-43. In one example, the PLD from a different RNA-binding protein is at least about 90%, at least about 95%, at least about 96%, at least about 97%, at least about 98%, at least about 99%, or 100% identical to the sequence set forth in SEQ ID NO: 13 or 27. In one example, the PLD from a different RNA-binding protein comprises the sequence set forth in SEQ ID NO: 13 or 27. In another example, the PLD from a different RNA-binding protein consists essentially of the sequence set forth in SEQ ID NO: 13 or 27. In another example, the PLD from a different RNA-binding protein consists of the sequence set forth in SEQ ID NO: 13 or 27. In one example, the TDP-43 variant is at least about 90%, at least about 95%, at least about 96%, at least about 97%, at least about 98%, at least about 99%, or 100% identical to the sequence set forth in SEQ ID NO: 28. In one example, the TDP-43 variant comprises the sequence set forth in SEQ ID NO: 28. In one example, the TDP-43 variant consists essentially of the sequence set forth in SEQ ID NO: 28. In one example, the TDP-43 variant consists of the sequence set forth in SEQ ID NO: 28.

In one example, the PLD from the wild type TDP-43 is replaced with a PLD from a different RNA-binding protein in the variant TDP-43. In one example, the PLD from a different RNA-binding protein is at least about 90%, at least about 95%, at least about 96%, at least about 97%, at least about 98%, at least about 99%, or 100% identical to the sequence set forth in SEQ ID NO: 58 or 68. In one example, the PLD from a different RNA-binding protein comprises the sequence set forth in SEQ ID NO: 58 or 68. In another example, the PLD from a different RNA-binding protein consists essentially of the sequence set forth in SEQ ID NO: 58 or 68. In another example, the PLD from a different RNA-binding protein consists of the sequence set forth in SEQ ID NO: 58 or 68. In one example, the TDP-43 variant is at least about 90%, at least about 95%, at least about 96%, at least about 97%, at least about 98%, at least about 99%, or 100% identical to the sequence set forth in SEQ ID NO: 69. In one example, the TDP-43 variant comprises the sequence set forth in SEQ ID NO: 69. In one example, the TDP-43 variant consists essentially of the sequence set forth in SEQ ID NO: 69. In one example, the TDP-43 variant consists of the sequence set forth in SEQ ID NO: 69.

In one example, the PLD from the wild type TDP-43 is replaced with a PLD from a different RNA-binding protein in the variant TDP-43. In one example, the PLD from a different RNA-binding protein is at least about 90%, at least about 95%, at least about 96%, at least about 97%, at least about 98%, at least about 99%, or 100% identical to the sequence set forth in SEQ ID NO: 21. In one example, the PLD from a different RNA-binding protein comprises the sequence set forth in SEQ ID NO: 21. In another example, the PLD from a different RNA-binding protein consists essentially of the sequence set forth in SEQ ID NO: 21. In another example, the PLD from a different RNA-binding protein consists of the sequence set forth in SEQ ID NO: 21.

In one example, the PLD from the wild type TDP-43 is replaced with a PLD from a different RNA-binding protein in the variant TDP-43. In one example, the PLD from a different RNA-binding protein is at least about 90%, at least about 95%, at least about 96%, at least about 97%, at least about 98%, at least about 99%, or 100% identical to the sequence set forth in SEQ ID NO: 73. In one example, the PLD from a different RNA-binding protein comprises the sequence set forth in SEQ ID NO: 73. In another example, the PLD from a different RNA-binding protein consists essentially of the sequence set forth in SEQ ID NO: 73. In another example, the PLD from a different RNA-binding protein consists of the sequence set forth in SEQ ID NO: 73.

The different RNA-binding protein can be one that is less-aggregation prone than wild type TDP-43. Likewise, the different RNA-binding protein can be one that has more aromatic residues in the PLD and/or has more uniformly spaced aromatic residues in the PLD than wild type TDP-43.

One example of such an RNA-binding protein is heterogeneous nuclear ribonucleoproteins A2/B1 (hnRNP A2/B1 or hnRNPA2B1). hnRNPA2B1 is a heterogeneous nuclear ribonucleoprotein (hnRNP) that associates with nascent pre-mRNAs, packaging them into hnRNP particles. The hnRNP particle arrangement on nascent hnRNA is non-random and sequence-dependent and serves to condense and stabilize the transcripts and minimize tangling and knotting. Packaging plays a role in various processes such as transcription, pre-mRNA processing, RNA nuclear export, subcellular location, mRNA translation and stability of mature mRNAs. Human hnRNPA2B1 is assigned UniProt reference number P22626. The human gene encoding hnRNPA2B1 (HNRNPA2B1 or HNRPA2B1) is assigned NCBI GeneID 3181 and is found at location 7p15.2 on chromosome 7 (assembly: GRCh38.p14 (GCF_000001405.40); location: NC_000007.14 (26189927 . . . 26200746, complement)). At least two isoforms of human hnRNPA2B1 are known. The first isoform is assigned UniProt reference number P22626-1 and NCBI reference number NP_112533.1 (SEQ ID NO: 7). An exemplary coding sequence is assigned reference number CCDS43557.1 (SEQ ID NO: 8), and an exemplary mRNA (cDNA) sequence is assigned reference number NM_031243.3 (SEQ ID NO: 9). The second isoform is assigned UniProt reference number P22626-2 and NCBI reference number NP_002128.1 (SEQ ID NO: 10). An exemplary coding sequence is assigned reference number CCDS5397.1 (SEQ ID NO: 11), and an exemplary mRNA (cDNA) sequence is assigned reference number NM_002137.3 (SEQ ID NO: 12). The PLD of human hnRNPA2B1 is set forth in SEQ ID NO: 13 or 27.

Mouse hnRNPA2B1 is assigned UniProt reference number 088569. The mouse gene encoding hnRNPA2B1 (Hnrnpa2b1 or Hnrpa2b1) is assigned NCBI GeneID 53379 and is found at location 6; 6 B3 on chromosome 6 (assembly: GRCm39 (GCF_000001635.27); location: NC_000072.7 (51437414 . . . 51448054, complement)). At least three isoforms of mouse hnRNPA2B1 are known. The first isoform is assigned UniProt reference number 088569-1 and NCBI reference numbers NP_001361674.1 and XP_006506436.2 (SEQ ID NO: 48). An exemplary coding sequence is assigned reference number CCDS90046.1 (SEQ ID NO: 49), and exemplary mRNA (cDNA) sequences are assigned reference numbers XM_006506373.3 (SEQ ID NO: 50) and NM_001374745.1 (SEQ ID NO: 51). The second isoform is assigned UniProt reference number 088569-2 and NCBI reference number NP_058086.2 (SEQ ID NO: 52). An exemplary coding sequence is assigned reference number CCDS51774.1 (SEQ ID NO: 53), and an exemplary mRNA (cDNA) sequence is assigned reference number NM_016806.3 (SEQ ID NO: 54). The third isoform is assigned UniProt reference number 088569-3 and NCBI reference number NP_872591.1 (SEQ ID NO: 55). An exemplary coding sequence is assigned reference number CCDS51773.1 (SEQ ID NO: 56), and an exemplary mRNA (cDNA) sequence is assigned reference number NM_182650.4 (SEQ ID NO: 57). The PLD of mouse hnRNPA2B1 is set forth in SEQ ID NO: 58 or 68.

Another example of such an RNA-binding protein is heterogeneous nuclear ribonucleoprotein A1 (hnRNPA1). hnRNPA1 is involved in the packaging of pre-mRNA into hnRNP particles, transport of poly(A) mRNA from the nucleus to the cytoplasm and modulation of splice site selection. Human hnRNPA1 is assigned UniProt reference number P09651. The human gene encoding hnRNPA1 (HNRNPA1 or HNRPA1) is assigned NCBI GeneID 3178 and is found at location 12q13.13 on chromosome 12 (assembly: GRCh38.p14 (GCF_000001405.40); location: NC_000012.12 (54280726 . . . 54287087)). At least three isoforms of human hnRNPA1 are known. The first isoform is assigned UniProt reference number P09651-1 and NCBI reference number NP_112420.1 (SEQ ID NO: 14). An exemplary coding sequence is assigned reference number CCDS44909.1 (SEQ ID NO: 15), and an exemplary mRNA (cDNA) sequence is assigned reference number NM_031157.4 (SEQ ID NO: 16). The second isoform is assigned UniProt reference number P09651-2 and NCBI reference number NP_002127.1 (SEQ ID NO: 17). An exemplary coding sequence is assigned reference number CCDS41793.1 (SEQ ID NO: 18), and an exemplary mRNA (cDNA) sequence is assigned reference number NM_002136.4 (SEQ ID NO: 19). The PLD of human hnRNPA2B1 (second isoform) is set forth in SEQ ID NO: 21. The third isoform is assigned UniProt reference number P09651-3 (SEQ ID NO: 20).

Mouse hnRNPA1 is assigned UniProt reference number P49312. The human gene encoding hnRNPA1 (Hnrnpa1 or Hnrpa1) is assigned NCBI GeneID 15382 and is found at location 15 F3; 15 58.58 cM on chromosome 15 (assembly: GRCm39 (GCF_000001635.27); location: NC_000081.7 (103148370 . . . 103155125)). At least two isoforms of mouse hnRNPA1 are known. The first isoform is assigned UniProt reference number P49312-1 and NCBI reference number NP_034577.1 (SEQ ID NO: 59). An exemplary coding sequence is assigned reference number CCDS37233.1 (SEQ ID NO: 60), and an exemplary mRNA (cDNA) sequence is assigned reference number NM_010447.5 (SEQ ID NO: 61). The second isoform is assigned UniProt reference number P49312-2 (SEQ ID NO: 62). The PLD of mouse hnRNPA2B1 is set forth in SEQ ID NO: 73.

In some cases, the TDP-43 variant is less prone to aggregation than wild type TDP-43. In some cases, the TDP-43 variant retains functions of wild type TDP-43 in splicing regulation (e.g., retains most of the functionality of wild type TDP-43 in regulating splicing of Adnp2, Dnajc5, Tsn, and/or Sortilin1). In some cases, the TDP-43 variant is predominantly nuclear. In some cases, the TDP-43 variant retains the subcellular distribution of wild type TDP-43. In some cases, the TDP-43 variant retains the function of wild type TDP-43 during embryonic development.

### III. Nucleic Acids Encoding TDP-43 Variants

Provided herein are nucleic acids or nucleic acid constructs encoding TDP-43 variants. The nucleic acids or nucleic acid constructs can be isolated nucleic acid constructs.

In some cases, the nucleic acid encoding the TDP-43 variant can be codon-optimized (e.g., codon-optimized for expression in a human or expression in a mouse). For example, the nucleic acid can be modified to substitute codons having a higher frequency of usage in a human cell, a non-human cell, a mammalian cell, a rodent cell, a mouse cell, a rat cell, or any other host cell of interest.

The nucleic acid encoding the TDP-43 variant can be DNA or RNA. The nucleic acid in some cases can be a messenger RNA (mRNA) encoding the TDP-43 variant. The nucleic acid in some cases can be a complementary DNA (cDNA) encoding the TDP-43 variant. Examples of coding sequences for some of the TDP-43 variants described herein are set forth, e.g., in SEQ ID NOS: 70-72. For example, such nucleic acids may contain only coding sequence without any intervening introns. In other cases, the nucleic acid can comprise one or more introns separating exons in the TDP-43 variant coding sequence. For example, the nucleic acid can comprise genomic sequence including both exons and introns.

In some cases, the nucleic acid is in an expression construct comprising the nucleic acid encoding the TDP-43 variant operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In a specific example, the promoter is active in a glial cell. In a specific example, the promoter is active in a muscle cell. In some cases, the promoter is a heterologous promoter (i.e., a promoter to which a TDP-43 nucleic acid is not operably linked naturally). In other cases, the promoter can be an endogenous promoter (i.e., TDP-43 variant nucleic acid operably linked to a TDP-43 promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter). For example, the promoter can be a glial-specific promoter. For example, the promoter can be a muscle-specific promoter.

The nucleic acids and expression constructs disclosed herein can also comprise post-transcriptional regulatory elements, such as the woodchuck hepatitis virus post-transcriptional regulatory element.

The nucleic acids and expression constructs can further comprise one or more polyadenylation signal sequences. For example, the nucleic acid construct can comprise a polyadenylation signal sequence located 3′ of the TDP-43 variant coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells.

The nucleic acids and expression constructs can also comprise a polyadenylation signal sequence upstream of the TDP-43 variant coding sequence. The polyadenylation signal sequence upstream of the TDP-43 variant coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the TDP-43 variant encoded by the nucleic acid or expression constructs can be expressed in a neuron-specific manner. In one example, the TDP-43 variant encoded by the nucleic acid or expression constructs can be expressed in a glial-specific manner. In one example, the TDP-43 variant encoded by the nucleic acid or expression constructs can be expressed in a muscle-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). For example, modifications can be made to one or more nucleosides within an mRNA. Examples of chemical modifications to mRNA nucleobases include pseudouridine, 1-methyl-pseudouridine, and 5-methyl-cytidine. mRNA can also be capped. mRNA can also be polyadenylated (to comprise a poly(A) tail). As one example, capped and polyadenylated mRNA containing N1-methyl-pseudouridine can be used (e.g., can be fully substituted with N1-methyl-pseudouridine). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo), hygromycin B phosphotransferase (hyg), puromycin-N-acetyltransferase (puro), blasticidin S deaminase (bsr), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

### IV. Vectors

Also provided herein are vectors comprising the nucleic acids, nucleic acid constructs, or expression constructs encoding TDP-43 variants. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The nucleic acids or expression constructs can be in a vector, such as a viral vector. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include about 10¹², about 10¹³, about 10¹⁴, about 10¹⁵, and about 10¹⁶ vector genomes/mL. Other exemplary viral titers (e.g., AAV titers) include about 10¹², about 10¹³, about 10¹⁴, about 10¹⁵, and about 10¹⁶ vector genomes (vg)/kg of body weight.

In one example, the nucleic acid or expression construct is in an AAV vector. The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used. Serotypes for use in skeletal muscle transduction include, for example, AAV1, AAV2, AAV6, and AAV9. See, e.g., Riaz et al. (2015) Skeletal Muscle Vol. 5, Article 37, herein incorporated by reference in its entirety for all purposes.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### V. Lipid Nanoparticles

Also provided herein are lipid nanoparticles comprising the TDP-43 variant or the nucleic acids, nucleic acid constructs, expression constructs, or vectors encoding the TDP-43 variant.

Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

### VI. Compositions

Also provided herein are compositions comprising the TDP-43 variants or the nucleic acids, nucleic acid constructs, expression constructs, vectors, or lipid nanoparticle disclosed herein. Such compositions can be, for example, for use in administering TDP-43 variants into a cell or subject or for use in expressing the TDP-43 variants in a cell or subject. Such compositions can be, for example, for use in inhibiting or decreasing TDP-43 aggregation in a cell or subject. Such compositions can be, for example, for use in rescuing aberrant TDP-43 splicing regulation in a cell or subject. Such compositions can be, for example, for use in rescuing aberrant subcellular distribution of endogenous TDP-43 in a cell or a subject. Such compositions can be, for example, for use in treating a TDP-43 proteinopathy in a subject. Such compositions can be, for example, for use in preventing a TDP-43 proteinopathy in a subject.

### VI. Cells or Animals

Cells or subjects (e.g., animals or non-human animals) comprising the TDP-43 variants or the nucleic acids, nucleic acid constructs, expression constructs, vectors, or lipid nanoparticles disclosed herein are also provided. The cells or subjects can express the TDP-43 variants.

The cells or subjects can be, for example, mammalian, non-human mammalian, and human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans.

The cells can be isolated cells (e.g., in vitro) or can be in vivo within a subject (e.g., animal or mammal). Cells can also be any type of undifferentiated or differentiated state. In one example, the cells are neurons. In one example, the cells are glial cells. In one example, the cells are muscle cells.

The cells provided herein can be normal, healthy cells, or can be diseased cells comprising TDP-43 aggregates or aberrant TDP-43 function (e.g., aberrant TDP-43 splicing regulation or aberrant TDP-43 subcellular localization). The cells can be, for example, prone to TDP-43 aggregation, or they can have preexisting TDP-43 aggregation.

In one example, the cell is a human cell, a rodent cell, a mouse cell, or a rat cell such as a human neuron, a rodent neuron, a mouse neuron, or a rat neuron, or a human glial cell, a rodent glial cell, a mouse glial cell, or a rat glial cell, or a human muscle cell, a rodent muscle cell, a mouse muscle cell, or a rat muscle cell. In a specific example, the cell is a human neuron. In a specific example, the cell is a human glial cell. In a specific example, the cell is a human muscle cell. In a specific example, the cell is in vivo in a subject (e.g., a neuron in the brain of a subject or a glial cell in the brain of a subject or a muscle cell in a subject).

In some such cells or animals, endogenous TDP-43 is not expressed in the cell or animal. For example, the endogenous TARDBP genomic locus can comprise a mutation that prevents expression of endogenous TDP-43 in the cell or the animal. Similarly, the cell or the animal can comprise an agent that reduces or eliminates expression of endogenous TDP-43 in the cell. Such agents are described in more detail below. For example, the agent can comprise an antisense oligonucleotide or an RNAi agent targeting endogenous TARDBP messenger RNA or a nucleic acid encoding the antisense oligonucleotide or the RNAi agent. The agent can also comprise a nuclease agent targeting the endogenous TARDBP genomic locus or one or more nucleic acids encoding the nuclease agent. For example, the nuclease agent can be a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In a specific example, the nuclease agent is the Cas protein and the guide RNA (e.g., a Cas9 protein and a guide RNA). Other examples of suitable agents are described in more detail elsewhere herein.

Some cells or animals have a genetically modified endogenous TARDBP genomic locus, wherein the nucleic acid encoding the TDP-43 variant is integrated at the endogenous TARDBP genomic locus. The cells or animals can be heterozygous or homozygous for the integrated nucleic acid. The integrated nucleic acid can be operably linked to the endogenous TARDBP promoter, or it can be operably linked to an exogenous promoter. In one example, the TDP-43 variant is expressed from the endogenous TARDBP genomic locus and replaces expression of the endogenous TDP-43.

Such cells or animals can have any of the phenotypes described herein that are associated with the TDP-43 variants. For example, the cell or animal can have reduced TDP-43 aggregation compared to a control cell without the TDP-43 variant or the nucleic acid.

Such cells or animals can be made by any suitable method. For example, a method of making such cells or animals can comprise administering the TDP-43 variant or the nucleic acid to the cell or animal. Suitable methods of administration are described in more detail elsewhere herein.

Various methods are provided for making a non-human animal genome, non-human animal cell, or non-human animal comprising a genetically modified endogenous TARDBP genomic locus as disclosed elsewhere herein. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted TARDBP locus.

For example, the method of producing a non-human animal comprising a genetically modified endogenous TARDBP genomic locus can comprise: (1) modifying the genome of a pluripotent cell to comprise the genetically modified endogenous TARDBP genomic locus; (2) identifying or selecting the genetically modified pluripotent cell comprising the genetically modified endogenous TARDBP genomic locus; (3) introducing the genetically modified pluripotent cell into a non-human animal host embryo; and (4) gestating the host embryo in a surrogate mother. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the genetically modified endogenous TARDBP genomic locus.

The methods can further comprise identifying a cell or animal having a modified target genomic locus. Various methods can be used to identify cells and animals having a targeted genetic modification.

The step of modifying the genome can, for example, utilize exogenous donor nucleic acids (e.g., targeting vectors) to modify a TARDBP locus to comprise a genetically modified endogenous TARDBP genomic locus disclosed herein. As one example, the targeting vector can be for generating a genetically modified endogenous TARDBP genomic locus, wherein the targeting vector comprises a 5′ homology arm targeting a 5′ target sequence at the endogenous TARDBP locus and a 3′ homology arm targeting a 3′ target sequence at the endogenous TARDBP locus. Exogenous donor nucleic acids can also comprise nucleic acid inserts including segments of DNA to be integrated in the locus. Integration of a nucleic acid insert in the TARDBP locus can result in addition of a nucleic acid sequence of interest in the TARDBP locus, deletion of a nucleic acid sequence of interest in the TARDBP locus, or replacement of a nucleic acid sequence of interest in the TARDBP locus (i.e., deletion and insertion). The homology arms can flank an insert nucleic acid comprising the nucleic acid encoding the TDP-43 variant to generate the genetically modified endogenous TARDBP genomic locus.

The exogenous donor nucleic acids can be for non-homologous-end-joining-mediated insertion or homologous recombination. Exogenous donor nucleic acids can comprise deoxyribonucleic acid (DNA) or ribonucleic acid (RNA), they can be single-stranded or double-stranded, and they can be in linear or circular form. For example, a repair template can be a single-stranded oligodeoxynucleotide (ssODN).

Exogenous donor nucleic acids can also comprise a heterologous sequence that is not present at an untargeted endogenous TARDBP locus. For example, an exogenous donor nucleic acids can comprise a selection cassette, such as a selection cassette flanked by recombinase recognition sites.

Some exogenous donor nucleic acids comprise homology arms. If the exogenous donor nucleic acid also comprises a nucleic acid insert, the homology arms can flank the nucleic acid insert. For ease of reference, the homology arms are referred to herein as 5′ and 3′ (i.e., upstream and downstream) homology arms. This terminology relates to the relative position of the homology arms to the nucleic acid insert within the exogenous donor nucleic acid. The 5′ and 3′ homology arms correspond to regions within the TARDBP locus, which are referred to herein as “5′ target sequence” and “3′ target sequence,” respectively.

A homology arm and a target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. In some targeting vectors, the intended mutation in the endogenous TARDBP locus is included in an insert nucleic acid flanked by the homology arms.

In cells other than one-cell stage embryos, the exogenous donor nucleic acid can be a “large targeting vector” or “LTVEC,” which includes targeting vectors that comprise homology arms that correspond to and are derived from nucleic acid sequences larger than those typically used by other approaches intended to perform homologous recombination in cells. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb.

The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). The modified pluripotent cell can be generated, for example, through recombination by (a) introducing into the cell one or more exogenous donor nucleic acids (e.g., targeting vectors) comprising an insert nucleic acid flanked, for example, by 5′ and 3′ homology arms corresponding to 5′ and 3′ target sites, wherein the insert nucleic acid comprises the nucleic acid encoding the TDP-43 variant to generate a genetically modified endogenous TARDBP genomic locus; and (b) identifying at least one cell comprising in its genome the insert nucleic acid integrated at the endogenous TARDBP locus (i.e., identifying at least one cell comprising the genetically modified endogenous TARDBP genomic locus).

Alternatively, the modified pluripotent cell can be generated by (a) introducing into the cell: (i) a nuclease agent or a nucleic acid encoding the nuclease agent, wherein the nuclease agent induces a nick or double-strand break at a target site within the endogenous TARDBP locus; and (ii) one or more exogenous donor nucleic acids (e.g., targeting vectors) comprising an insert nucleic acid flanked by, for example, 5′ and 3′ homology arms corresponding to 5′ and 3′ target sites located in sufficient proximity to the nuclease target site, wherein the insert nucleic acid comprises a nucleic acid encoding a TDP-43 variant to generate a genetically modified endogenous TARDBP genomic locus; and (c) identifying at least one cell comprising in its genome the insert nucleic acid integrated at the endogenous TARDBP locus (i.e., identifying at least one cell comprising the genetically modified endogenous TARDBP genomic locus). Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes.

The donor cell can be introduced into a host embryo at any stage, such as the blastocyst stage or the pre-morula stage (i.e., the 4 cell stage or the 8 cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the genetically modified endogenous TARDBP genomic locus; (2) selecting the genetically modified embryo; and (3) gestating the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the genetically modified endogenous TARDBP genomic locus. It is recognized that depending on the method used to generate the F0 animal, the number of cells within the F0 animal that have the genetically modified endogenous TARDBP genomic locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for the genetically modified endogenous TARDBP genomic locus or can be homozygous for the genetically modified endogenous TARDBP genomic locus.

### VIII. Methods

Provided herein are methods of administering a TDP-43 variant as described herein to a cell or subject, or administering a nucleic acid encoding the TDP-43 variant to the cell or the subject such that the TDP-43 variant is expressed. Also provided are methods of reducing or inhibiting TDP-43 aggregation in a cell or a subject, methods of rescuing aberrant TDP-43 splicing regulation in a cell or a subject, or methods of rescuing aberrant subcellular distribution of TDP-43 in a cell or a subject. Also provided are methods of treating a TDP-43 proteinopathy in a subject or methods of preventing a TDP-43 proteinopathy in a subject. Such methods can comprise administering a TDP-43 variant (e.g., a therapeutically effective amount) as described herein to a cell or subject, or administering a nucleic acid encoding the TDP-43 variant (e.g., a therapeutically effective amount) to the cell or the subject such that the TDP-43 variant is expressed. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Some such methods comprise administering the TDP-43 variant to the cell or the subject. Some such methods comprise administering the nucleic acid encoding the TDP-43 variant to the cell or the subject. The nucleic acid can be a nucleic acid construct described in more detail elsewhere herein. In some cases, the nucleic acid encoding the TDP-43 variant can be codon-optimized (e.g., codon-optimized for expression in a human or expression in a mouse). For example, the nucleic acid can be modified to substitute codons having a higher frequency of usage in a human cell, a non-human cell, a mammalian cell, a rodent cell, a mouse cell, a rat cell, or any other host cell of interest.

The nucleic acid encoding the TDP-43 variant can be DNA or RNA. The nucleic acid in some cases can be a messenger RNA (mRNA) encoding the TDP-43 variant. The nucleic acid in some cases can be a complementary DNA (cDNA) encoding the TDP-43 variant. For example, such nucleic acids may contain only coding sequence without any intervening introns. Examples of coding sequences for some of the TDP-43 variants described herein are set forth, e.g., in SEQ ID NOS: 70-72. In other cases, the nucleic acid can comprise one or more introns separating exons in the TDP-43 variant coding sequence. For example, the nucleic acid can comprise TDP-43 variant genomic sequence including both exons and introns.

In some methods, the nucleic acid is in an expression construct comprising the nucleic acid encoding the TDP-43 variant operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In a specific example, the promoter is active in a glial cell. In a specific example, the promoter is active in a muscle cell. In some cases, the promoter is a heterologous promoter (i.e., a promoter to which a TDP-43 nucleic acid is not naturally operably linked). In other cases, the promoter can be an endogenous promoter (i.e., TDP-43 variant nucleic acid operably linked to a TARDBP promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter that is very specific with a low level of expression is a synapsin-1 promoter (e.g., a human synapsin-1 promoter). For example, the promoter can be a glial-specific promoter. For example, the promoter can be a muscle-specific promoter.

The nucleic acids and expression constructs disclosed herein can also comprise post-transcriptional regulatory elements, such as the woodchuck hepatitis virus post-transcriptional regulatory element. For example, the promoter can be a glial-specific promoter.

The nucleic acids and expression constructs can further comprise one or more polyadenylation signal sequences. For example, the nucleic acid construct can comprise a polyadenylation signal sequence located 3′ of the TDP-43 variant coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells.

The nucleic acids and expression constructs can also optionally comprise a polyadenylation signal sequence upstream of the TDP-43 variant coding sequence. The polyadenylation signal sequence upstream of the TDP-43 variant coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the TDP-43 variant encoded by the nucleic acid or expression constructs can be expressed in a neuron-specific manner. In one example, the TDP-43 variant encoded by the nucleic acid or expression constructs can be expressed in a glial-specific manner. In one example, the TDP-43 variant encoded by the nucleic acid or expression constructs can be expressed in a muscle-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). For example, modifications can be made to one or more nucleosides within an mRNA. Examples of chemical modifications to mRNA nucleobases include pseudouridine, 1-methyl-pseudouridine, and 5-methyl-cytidine. mRNA can also be capped. mRNA can also be polyadenylated (to comprise a poly(A) tail). As one example, capped and polyadenylated mRNA containing N1-methyl-pseudouridine can be used (e.g., can be fully substituted with N1-methyl-pseudouridine). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo), hygromycin B phosphotransferase (hyg), puromycin-N-acetyltransferase (puro), blasticidin S deaminase (bsr), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

The nucleic acids or expression constructs can be in a vector, such as a viral vector. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The nucleic acids or expression constructs can be in a vector, such as a viral vector. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include about 10¹², about 10¹³, about 10¹⁴, about 10¹⁵, and about 10¹⁶ vector genomes/mL. Other exemplary viral titers (e.g., AAV titers) include about 10¹², about 10¹¹, about 10¹⁴, about 10¹⁵, and about 10¹⁶ vector genomes (vg)/kg of body weight.

In one example, the nucleic acid or expression construct is in an AAV vector. The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used. Serotypes for use in skeletal muscle transduction include, for example, AAV1, AAV2, AAV6, and AAV9. See, e.g., Riaz et al. (2015) Skeletal Muscle Vol. 5, Article 37, herein incorporated by reference in its entirety for all purposes.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In some methods, the TDP-43 variant or the nucleic acid encoding the TDP-43 variant is associated with a lipid nanoparticle. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

The TDP-43 variant, or the nucleic acid encoding the TDP-43 variant can be administered to the cell or the subject by any suitable means. Various methods and compositions are provided herein to allow for introduction of molecule (e.g., a nucleic acid or protein) into a cell or subject.

The methods provided herein do not depend on a particular method for introducing a nucleic acid or protein into the cell, only that the nucleic acid or protein gains access to the interior of the cell. Methods for introducing nucleic acids and proteins into various cell types are known in the art and include, for example, stable transfection methods, transient transfection methods, and virus-mediated methods.

Transfection protocols as well as protocols for introducing molecules (e.g., nucleic acids or proteins) into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4): 1590-4, and Kriegler, M (1991). Transfer and Expression: A Laboratory Manual. New York: W. H. Freeman and Company. pp. 96-97, each of which is herein incorporated by reference in its entirety for all purposes); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28, herein incorporated by reference in its entirety for all purposes). Viral methods can also be used for transfection.

Introduction of molecules (e.g., nucleic acids or proteins) into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell can also be accomplished by microinjection. Microinjection of an mRNA is preferably into the cytoplasm (e.g., to deliver mRNA directly to the translation machinery), while microinjection of a protein or a DNA encoding a protein is preferably into the nucleus. Alternatively, microinjection can be carried out by injection into both the nucleus and the cytoplasm: a needle can first be introduced into the nucleus and a first amount can be injected, and while removing the needle from the cell a second amount can be injected into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acids or proteins) into a cell can include, for example, vector delivery, particle-mediated delivery, exosome-mediated delivery, lipid-nanoparticle-mediated delivery, cell-penetrating-peptide-mediated delivery, or implantable-device-mediated delivery. Methods of administering nucleic acids or proteins to a subject to modify cells in vivo are disclosed elsewhere herein. As specific examples, a molecule (e.g., nucleic acid or protein) can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., lentivirus-mediated delivery or adeno-associated virus (AAV)-mediated delivery), and lipid-nanoparticle-mediated delivery.

In one example, the TDP-43 variant, or the nucleic acid encoding the TDP-43 variant can be administered via viral transduction such as lentiviral transduction or adeno-associated viral transduction. In another example, the TDP-43 variant, or the nucleic acid encoding the TDP-43 variant can be administered via lipid nanoparticle (LNP)-mediated delivery.

Administration in vivo can be by any suitable route such that the TDP-43 variant, or the nucleic acid encoding the TDP-43 variant reaches the intended target cell(s) (e.g., neurons in the brain of the subject and/or glial cells in the brain of the subject and/or muscle cells of the subject) or target tissue (e.g., brain or muscle). Examples of routes of administration include parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. For example, the TDP-43 variant, or the nucleic acid encoding the TDP-43 variant may be administered directly to the brain of a subject or to neurons or glial cells in the brain of a subject. In a specific example, administration to a subject is by intrathecal injection or by intracranial injection (e.g., stereotactic surgery for injection in the hippocampus and other brain regions, or intracerebroventricular injection). In a specific example, administration to a subject is by intracerebroventricular injection. In another specific example, administration to a subject is by intracranial injection. In another specific example, administration to a subject is by intrathecal injection. In another example, the TDP-43 variant, or the nucleic acid encoding the TDP-43 variant may be administered directly to the muscle of a subject or to muscle cells in a subject (e.g., intramuscular injection).

The frequency of administration and the number of dosages can depend on the half-life of the composition being administered and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

The cells or subjects in the methods can be, for example, mammalian, non-human mammalian, and human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans. In a specific example, the cells or subjects are human.

The cells can be isolated cells (e.g., in vitro) or can be in vivo within a subject (e.g., animal or mammal). Cells can also be any type of undifferentiated or differentiated state. In one example, the cells are neurons. In one example, the cells are glial cells. In one example, the cells are muscle cells.

The cells provided herein can be normal, healthy cells, or can be diseased cells comprising TDP-43 aggregates or aberrant TDP-43 function (e.g, aberrant TDP-43 splicing regulation or aberrant TDP-43 subcellular localization). The cells can be, for example, prone to TDP-43 aggregation, or they can have preexisting TDP-43 aggregation.

In one example, the cell is a human cell, a rodent cell, a mouse cell, or a rat cell such as a human neuron, a rodent neuron, a mouse neuron, or a rat neuron, or a human glial cell, a rodent glial cell, a mouse glial cell, or a rat glial cell, or a human muscle cell, a rodent muscle cell, a mouse muscle cell, or a rat muscle cell. In a specific example, the cell is a human neuron. In a specific example, the cell is a human glial cell. In a specific example, the cell is a human muscle cell. In a specific example, the cell is in vivo in a subject (e.g., a neuron or glial cell in the brain of a subject or a muscle cell in a subject). For example, such methods can be methods of inhibiting or reducing TDP-43 aggregation, methods of reducing TDP-43 phosphorylation, methods of reducing or rescuing aberrant regulation of splicing by TDP-43, or methods of reducing or rescuing aberrant TDP-43 subcellular localization (e.g., reducing or rescuing aberrant nuclear depletion of TDP-43) in a cell of a subject (e.g., a neuron or glial cell in the brain of the subject or a muscle cell in the subject).

The methods described herein can further comprise administering to the cell or subject an agent that reduces or eliminates expression of endogenous TDP-43 in the cell or subject. Any suitable agent can be used to reduce or inhibit expression of endogenous TDP-43. Examples of agents that can reduce expression of endogenous TDP-43 include nuclease agents (e.g., ZFNs, TALENs, or CRISPR/Cas), DNA-binding proteins fused to a transcriptional repressor (e.g., transcriptional repressors such as a catalytically inactive/dead Cas (dCas) fused to a KRAB domain (dCas-KRAB)), or antisense oligonucleotides, siRNAs, shRNAs, or antisense RNAs.

Nuclease agents can be used to decrease expression of endogenous TDP-43. For example, such nuclease agents can be designed to target and cleave a region of a TARDBP gene that will disrupt expression of the TARDBP gene. As a specific example, a nuclease agent can be designed to cleave a region of a TARDBP near the start codon. For example, the target sequence can be within about 10, 20, 30, 40, 50, 100, 200, 300, 400, 500, or 1,000 nucleotides of the start codon, and cleavage by the nuclease agent can disrupt the start codon. Alternatively, nuclease agents designed to cleave regions near the start and stop codons can be used in order to delete the coding sequence between the two nuclease target sequences. DNA-binding proteins fused to transcriptional repressor domains can also be used to decrease expression of endogenous TDP-43. For example, a DNA-binding protein fused to a transcriptional repressor domain (e.g., catalytically inactive Cas fused to a KRAB transcriptional repressor domain) can be designed to target a region of TARDBP near the start codon e.g., within about 10, 20, 30, 40, 50, 100, 200, 300, 400, 500, or 1,000 nucleotides of the start codon).

Cleavage by a nuclease agent can result in a double-strand break that can be repaired by non-homologous end joining (NHEJ). NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. These insertions and deletions (indels) can disrupt expression of the target gene through, for example, frameshift mutations or disruption of the start codon.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” includes a nuclease that is engineered (modified or derived) from its native form to specifically recognize and induce a nick or double-strand break in the desired recognition site. Thus, an engineered nuclease agent can be derived from a native, naturally occurring nuclease agent or it can be artificially created or synthesized. The engineered nuclease can induce a nick or double-strand break in a recognition site, for example, wherein the recognition site is not a sequence that would have been recognized by a native (non-engineered or non-modified) nuclease agent. The modification of the nuclease agent can be as little as one amino acid in a protein cleavage agent or one nucleotide in a nucleic acid cleavage agent. Producing a nick or double-strand break in a recognition site or other DNA can be referred to herein as “cutting” or “cleaving” the recognition site or other DNA.

Active variants and fragments of the exemplified recognition sites are also provided. Such active variants can comprise at least 65%, 70%, 75%, 80%, 85%, 90%, 91%, 92%, 93%, 94%, 95%, 96%, 97%, 98%, 99% or more sequence identity to the given recognition site, wherein the active variants retain biological activity and hence are capable of being recognized and cleaved by a nuclease agent in a sequence-specific manner. Assays to measure the double-strand break of a recognition site by a nuclease agent are known in the art (e.g., TaqMan® qPCR assay, Frendewey et al. (2010) Methods in Enzymology 476:295-307, herein incorporated by reference in its entirety for all purposes).

One type of nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See WO 2010/079430; Morbitzer et al. (2010) Proc. Natl. Acad. Sci. U.S.A. 107(50):21617-21622; Scholze & Boch (2010) Virulence 1:428-432; Christian et al. Genetics (2010) 186:757-761; Li et al. (2010) Nucleic Acids Res. (2011) 39(1):359-372; and Miller et al. (2011) Nature Biotechnology 29:143-148, each of which is herein incorporated by reference in its entirety for all purposes.

Examples of suitable TAL nucleases, and methods for preparing suitable TAL nucleases, are disclosed, e.g., in US 2011/0239315 A1, US 2011/0269234 A1, US 2011/0145940 A1, US 2003/0232410 A1, US 2005/0208489 A1, US 2005/0026157 A1, US 2005/0064474 A1, US 2006/0188987 A1, and US 2006/0063231 A1, each of which is herein incorporated by reference in its entirety for all purposes. In various embodiments, TAL effector nucleases are engineered that cut in or near a target nucleic acid sequence in, e.g., a locus of interest or a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In some TALENs, each monomer of the TALEN comprises 33-35 TAL repeats that recognize a single base pair via two hypervariable residues. In some TALENs, the nuclease agent is a chimeric protein comprising a TAL-repeat-based DNA binding domain operably linked to an independent nuclease such as a FokI endonuclease. For example, the nuclease agent can comprise a first TAL-repeat-based DNA binding domain and a second TAL-repeat-based DNA binding domain, wherein each of the first and the second TAL-repeat-based DNA binding domains is operably linked to a FokI nuclease, wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

The nuclease agent employed in the various methods and compositions disclosed herein can further comprise a zinc-finger nuclease (ZFN). In some ZFNs, each monomer of the ZFN comprises 3 or more zinc finger-based DNA binding domains, wherein each zinc finger-based DNA binding domain binds to a 3 bp subsite. In other ZFNs, the ZFN is a chimeric protein comprising a zinc finger-based DNA binding domain operably linked to an independent nuclease such as a FokI endonuclease. For example, the nuclease agent can comprise a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease that makes a double strand break. See, e.g., US20060246567; US20080182332; US20020081614; US20030021776; WO/2002/057308A2; US20130123484; US20100291048; WO/2011/017293A2; and Gaj et al. (2013) Trends in Biotechnology, 31(7):397-405, each of which is herein incorporated by reference in its entirety for all purposes.

The methods and compositions disclosed herein can utilize Clustered Regularly Interspersed Short Palindromic Repeats (CRISPR)/CRISPR-associated (Cas) systems or components of such systems to modify a genome or alter expression of a gene within a cell. CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes.

CRISPR/Cas systems used in the compositions and methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

Active variants and fragments of nuclease agents (i.e., an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus, some engineered nucleases have a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

The nuclease agent may be introduced into the cell by any known means. The polypeptide encoding the nuclease agent may be directly introduced into the cell. Alternatively, a one or more nucleic acids encoding the nuclease agent can be introduced into the cell. When a nucleic acid encoding the nuclease agent is introduced into the cell, the nuclease agent can be transiently, conditionally, or constitutively expressed within the cell. Thus, the nucleic acid(s) encoding the nuclease agent can be contained in an expression cassette and be operably linked to a conditional promoter, an inducible promoter, a constitutive promoter, or a tissue-specific promoter. Such promoters of interest are discussed in further detail elsewhere herein. Alternatively, the nuclease agent is introduced into the cell as an mRNA encoding a nuclease agent.

A nucleic acid encoding a nuclease agent can be stably integrated in the genome of the cell and operably linked to a promoter active in the cell. Alternatively, a nucleic acid encoding a nuclease agent can be in a targeting vector (e.g., a targeting vector comprising an insert polynucleotide, or in a vector or a plasmid that is separate from the targeting vector comprising the insert polynucleotide).

When the nuclease agent is provided to the cell through the introduction of nucleic acid(s) encoding the nuclease agent, such a nucleic acid encoding a nuclease agent can be modified to substitute codons having a higher frequency of usage in the cell of interest, as compared to the naturally occurring polynucleotide sequence encoding the nuclease agent. For example, the polynucleotide encoding the nuclease agent can be modified to substitute codons having a higher frequency of usage in a given prokaryotic or eukaryotic cell of interest, including a bacterial cell, a yeast cell, a human cell, a non-human cell, a mammalian cell, a rodent cell, a mouse cell, a rat cell or any other host cell of interest, as compared to the naturally occurring polynucleotide sequence.

Antisense oligonucleotides, antisense RNAs, small interfering RNAs (siRNAs), or short hairpin RNAs (shRNAs) can also be used to decrease expression of endogenous TDP-43. Such antisense RNAs, siRNAs, or shRNAs can be designed to target any region of a TARDBP mRNA.

The term “antisense RNA” refers to a single-stranded RNA that is complementary to a messenger RNA strand transcribed in a cell. The term “small interfering RNA (siRNA)” refers to a typically double-stranded RNA molecule that induces the RNA interference (RNAi) pathway. These molecules can vary in length (generally between 18-30 base pairs) and contain varying degrees of complementarity to their target mRNA in the antisense strand. Some, but not all, siRNAs have unpaired overhanging bases on the 5′ or 3′ end of the sense strand and/or the antisense strand. The term “siRNA” includes duplexes of two separate strands, as well as single strands that can form hairpin structures comprising a duplex region. The double-stranded structure can be, for example, less than 20, 25, 30, 35, 40, 45, or 50 nucleotides in length. For example, the double-stranded structure can be from about 21-23 nucleotides in length, from about 19-25 nucleotides in length, or from about 19-23 nucleotides in length. The term “short hairpin RNA (shRNA)” refers to a single strand of RNA bases that self-hybridizes in a hairpin structure and can induce the RNA interference (RNAi) pathway upon processing. These molecules can vary in length (generally about 50-90 nucleotides in length, or in some cases up to greater than 250 nucleotides in length, e.g., for microRNA-adapted shRNA). shRNA molecules are processed within the cell to form siRNAs, which in turn can knock down gene expression. shRNAs can be incorporated into vectors. The term “shRNA” also refers to a DNA molecule from which a short, hairpin RNA molecule may be transcribed.

Antisense oligonucleotides and RNAi agents can also be used to decrease expression of endogenous TDP-43. Such antisense oligonucleotides or RNAi agents can be designed to target any region of a TARDBP mRNA.

An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to, short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Single-stranded antisense oligonucleotides (ASOs) and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H.

ASOs are DNA oligos, typically 15-25 bases long, designed in antisense orientation to the RNA of interest. Examples of ASOs targeting TARDBP are provided, e.g., in US 2020-0165610, herein incorporated by reference in its entirety for all purposes. Hybridization of the ASO to the target RNA mediates RNase H cleavage of the RNA, which can prevent protein translation of the mRNA. To increase nuclease resistance, phosphorothioate (PS) modifications can be added to the oligo. Phosphorothioate linkages also promote binding to serum proteins, which increases the bioavailability of the ASO and facilitates productive cellular uptake. In phosphorothioates, a sulfur atom replaces a non-bridging oxygen in the oligo phosphate backbone. ASOs can be chimeras comprising both DNA and modified RNA bases. The use of modified RNA, such as 2′-O-methoxy-ethyl (2′-MOE) RNA, 2′-O-methyl (2′OMe) RNA, or Affinity Plus Locked Nucleic Acid bases in chimeric antisense designs, increases both nuclease stability and affinity (Tₘ) of the antisense oligo to the target RNA. However, these modifications do not activate RNase H cleavage (i.e., ASOs fully composed of sugar-modified RNA-like nucleotides (such as 2′-MOE), however, do not support RNase H cleavage of the complementary RNA). Thus, one antisense strategy is a “gapmer” design that incorporates 2′-O-modified RNA or Affinity Plus Locked Nucleic Acid bases in chimeric antisense oligos that retain an RNase-H-activating domain. A standard gapmer retains a central region of PS-modified DNA bases sufficient to induce RNase H cleavage. These bases are flanked on both sides by blocks of 2′ modifications that will increase binding affinity to the target. For example, gapmers can contain a central section of deoxynucleotides that allows the induction of RNase H cleavage, with the central part being flanked by blocks of 2′-O-alkyl modified ribonucleotides that protect the central section from nuclease degradation. Once delivered to cells, ASOs enter the nucleus and bind to their complementary, endogenous RNA target. Hybridization of the ASO gapmers to target RNA forms a DNA:RNA heteroduplex in the central region, which becomes a substrate for cleavage by the enzyme RNase H1.

In one example, the agent that reduces or eliminates expression of endogenous TDP-43 comprises an antisense oligonucleotide or an RNAi agent targeting endogenous TARDBP messenger RNA or a nucleic acid encoding the antisense oligonucleotide or the RNAi agent. In another example, the agent comprises a nuclease agent targeting the endogenous TARDBP genomic locus or one or more nucleic acids encoding the nuclease agent. The nuclease agent can be, for example, a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA. In a specific example, the nuclease agent is the Cas protein and the guide RNA, optionally wherein the Cas protein is a Cas9 protein. For example, the methods described herein can further comprise administering the nucleic acid encoding the TDP-43 variant, wherein the nuclease agent cleaves the endogenous TARDBP genomic locus, the nucleic acid encoding the TDP-43 variant is inserted at or recombines with the cleaved endogenous TARDBP genomic locus, wherein the TDP-43 variant is expressed from the endogenous TARDBP genomic locus and replaces expression of the endogenous TDP-43.

Such methods can further comprise screening the cells or subjects to confirm the presence of the TDP-43 variant, or the nucleic acid encoding the TDP-43 variant. Screening the cells or subjects for the TDP-43 variant, or the nucleic acid encoding the TDP-43 variant can be performed by any known means. Such methods can further comprise screening the cells or subjects to confirm expression of the nucleic acid encoding the TDP-43 variant. Screening the cells or subjects for expression of the nucleic acid encoding the TDP-43 variant can be performed by any known means. For example, methods for measuring protein expression and for measuring expression of mRNA encoded by a coding sequence are well-known.

One example of an assay that can be used is the BASESCOPE™ RNA in situ hybridization (ISH) assay, which a method that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a 1 ZZ probe and it can differentially detect single nucleotide edits and mutations in intact fixed tissue.

As another example, reporter genes can be used for screening. For example, the nucleic acid encoding the TDP-43 variant can encode a TDP-43 variant fused to a reporter gene such as a fluorescent protein. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase.

As another example, selection markers can be used to screen for cells that have the TDP-43 variant, or the nucleic acid encoding the TDP-43 variant. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k).

The methods can further comprise assessing one or more signs or symptoms of TDP-43 proteinopathy by any suitable means. Examples of such signs and symptoms are discussed in more detail elsewhere herein and include, for example, TDP-43 hyperphosphorylation, TDP-43 aggregation, aberrant splicing regulation by TDP-43, and aberrant TDP-43 subcellular distribution (e.g., aberrant nuclear depletion). This can be done, for example, about 1 day, about 2 days, about 3 days, about 4 days, about 5 days, about 6 days, about 7 days, about 1 week, about 2 weeks, about 3 weeks, about 4 weeks, about 5 weeks, about 6 weeks, or longer after introducing the TDP-43 variant, or the nucleic acid encoding the TDP-43 variant. For example, the assessing can be done about 2 weeks to about 6 weeks or about 3 weeks to about 5 weeks after introducing the TDP-43 variant, or the nucleic acid encoding the TDP-43 variant.

The methods described herein can, for example, reduce the amount of new TDP-43 aggregate formation (new TDP-43 aggregation) in a cell or a subject and/or can reduce the amount of existing TDP-43 aggregate formation (preexisting TDP-43 aggregation) in a cell or a subject. For example, the methods described herein can prevent new TDP-43 aggregate formation and/or can reverse existing TDP-43 aggregate formation.

The methods described herein can, for example, reduce the amount of aberrant regulation of splicing by TDP-43 in a cell or a subject. For example, the methods described herein can rescue aberrant regulation of splicing by TDP-43.

The methods described herein can, for example, reduce aberrant subcellular localization of TDP-43 (e.g., reduce aberrant nuclear depletion of TDP-43) in a cell or a subject. For example, the methods described herein can rescue aberrant subcellular localization of TDP-43 (e.g., rescue aberrant nuclear depletion of TDP-43).

The methods described herein can, for example, reduce the levels of phospho-TDP-43 in a cell or a subject.

In some of the methods described herein, the methods are for treating or preventing TDP-43 proteinopathies in a subject. Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. The compositions disclosed herein may be administered to relieve or prevent or decrease the severity of one or more of the signs or symptoms of TDP-43 proteinopathy described in more detail elsewhere herein.

In some methods (e.g., methods for treating), the subject has one or more signs or symptoms of a TDP-43 proteinopathy. For example, the subject can have preexisting TDP-43 aggregate formation in one or more cells. TDP-43 proteinopathies are classified based upon the extent of modified TDP-43 inclusions and include a growing number of neurodegenerative diseases including amyotrophic lateral sclerosis (ALS), frontotemporal lobar degeneration with ubiquitin immunoreactive, tau negative inclusions (FTLD-U) and FTLD with motor neuron disease (FTLD-MND). In addition, TDP-43 inclusions have also been identified in a number of other neurodegenerative disorders including Alzheimer's disease, corticobasal degeneration, Lewy body related diseases and Pick's disease. TDP-43 proteinopathy is usually characterized by the presence of aberrant phosphorylation, ubiquitination, cleavage, and/or nuclear depletion of TDP-43 in neurons and glial cells. Various major neurodegenerative diseases display similar TDP-43 pathological manifestations in neurons and even glia, including the accumulation of detergent-resistant, ubiquitinated, or hyperphosphorylated TDP-43 inclusions in the cytoplasm, usually accompanied by the depletion of TDP-43 from the nucleus. These characteristics of TDP-43-related pathological features are usually referred to as TDP-43 proteinopathy. See, e.g., Gao et al. (2018) J. Neurochem. doi: 10.1111/jnc.14327, herein incorporated by reference in its entirety for all purposes.

TDP-43 proteinopathies encompass a wide range of neurodegenerative diseases and phenotypes, which may be inherited in a Mendelian pattern or be apparently sporadic. TDP-43 has been found to be aggregated in several diseases, including Alzheimer's disease (brain), LATE (brain), and inclusion body myositis (muscle). A large number of genes and diseases have been associated with TDP-43 proteinopathies (Table 2). See, e.g., de Boer et al. (2021) J. Neurol. Neurosurg. Psychiatry 92:86-95, herein incorporated by reference in its entirety for all purposes.

TABLE 2
Diseases associated with TDP-43 pathology.

| | | Co-Occurrence | |
|---|---|---|---|
| | Predominant | of TDP-43 | |
| Disease | Pathology | Pathology | Associated Genes |
| Classic ALS | TDP-43 | n.a. | ALS2, SETX, TARDBP, VAPB, ANG, UBQLN2, OPTN, |
| | | | PFN1, UNC13a, NEK1, C21orf2, SIGMAR1, DCTN1, |
| | | | MATR3, VCP, hnRNPA1/A2b1, NIPA1, TBK1, ATXN2, |
| | | | UBQLN2, SQSTM1 |
| Familial ALS- | SOD1 | Rarely | SOD1 |
| SOD1 | | | |
| Familial ALS-FUS | FUS | No | FUS |
| ALS-FTLD, ALS- | TDP-43 | n.a. | TARDBP, CHMP2b, TBK1, UBQLN2, SQSTM1, |
| ci/bi | | | DCTN1, UNC13a |
| Classic ALS, | TDP-43 | n.a. | C9orf72 |
| ALS-FTLD, FTLD | | | |
| MSP* | TDP-43 | n.a. | VCP, hnRNPA1, hnRNPA2b1, SQSTM1 |
| FTLD | TDP-43 | n.a. | CHMP2b, GRN, SQSTM1, OPTN, TBK1, ATXN2 |
| FTLD | FUS | No | — |
| FTLD | Tau | No | MAPT |
| Alzheimer's | β-Amyloid, | Yes | APOE, APP, PSEN1, PSEN2 |
| disease | tau | | |
| Dementia with | α-Synuclein | Yes | SNCA, APP, PSEN1/PSEN2, MAPT, GBA, APOE |
| Lewy bodies | | | |
| Parkinson disease | α-Synuclein | Yes | TARDBP, SNCA, Parkin, PINK1, DJ-1, LRRK2, |
| | | | ATP13A2, PLA2G6 |
| Huntington disease | Huntingtin | yes | Huntingtin |
| | protein | | |
| LATE/CARTS | TDP-43, HS | n.a. | GRN, TMEM106B, ABCC9, KCNMB2, APOE |
| CTE | Tau | Yes | — |
| Perry disease | TDP-43 | n.a. | DCTN1 |
| FOSMN | TDP-43 | n.a. | SOD1, SQSTM1, VCP, CHCHD10 |
| sIBM | TDP-43 | n.a. | — |
| PSP | Tau | Yes | MAPT, STX6, EIF2AK3 |
| CBD | Tau | Yes | MAPT |
| AGD | Tau | Yes | — |

*Multiple system proteinopathy-A familial disorder in which patients present with ALS, FTLD, inclusion body myositis, Paget's disease of the bone or combinations of these phenotypes.
ALS, amyotrophic lateral sclerosis; bi, behavioral impairment; CARTS, cerebral age-related TDP-43 with sclerosis; ci, cognitive impairment; CTE, chronic traumatic encephalopathy; FOSMN, facial onset sensory and motor neuronopathy; FTLD, frontotemporal lobar degeneration; HS, hippocampal sclerosis; LATE, limbic-predominant age-related TDP-43 encephalopathy; n.a., not applicable; PPA, primary progressive aphasia; sIBM, sporadic inclusion body myositis; TDP-43, TAR DNA-binding protein 43.

TDP-43 aggregation is evident in ˜97% of all amyotrophic lateral sclerosis (ALS) cases. These TDP-43 inclusions are evident in both demented and non-demented patients with ALS, and increase in density with disease evolution particularly the development of cognitive impairment. In ALS, three predominating cell-type specific patterns of TDP-43 pathology have been identified, including (1) glial (22% of cases); (2) mixed neuronal and glial (5900 of cases); (3) neuronal (7% of cases). The extent of TDP-43 pathology differentiates ALS-FTLD from ALS without FTLD, and the presence of TDP-43 pathology in extra-motor areas was associated with cognitive impairment in ALS, as can be assessed by the Edinburgh Cognitive and Behavioral ALS Screen (ECAS). TDP-43 pathology in the orbitofrontal, dorsolateral prefrontal, medial prefrontal cortices and ventral anterior cingulate were associated with executive dysfunction. Language dysfunction was associated with TDP-43 pathology in the inferior frontal gyrus, transverse temporal area, middle and inferior temporal gyri, as well as the angular gyrus. Verbal fluency dysfunction was associated with TDP-43 pathology in the prefrontal cortex, inferior frontal gyrus, ventral anterior cingulate and transverse temporal area. Behavioral abnormalities, however, were associated with TDP-43 pathology in the orbitofrontal and prefrontal cortices as well as the ventral anterior cingulate.

The methods described herein can alleviate one or more signs and symptoms of TDP-43 proteinopathy in a cell or subject. Some examples of signs and symptoms of TDP-43 proteinopathy at the cellular level include aberrant phosphorylation, ubiquitination, cleavage, and/or nuclear depletion of TDP-43 in neurons or glial cells or muscle cells, or accumulation of detergent-resistant, ubiquitinated, or hyperphosphorylated TDP-43 inclusions in the cytoplasm, usually accompanied by the depletion of TDP-43 from the nucleus. Other signs and symptoms at an organism level can include neurodegeneration, a term that refers to the progressive loss of structure and function of neurons.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 3
Description of Sequences.

| SEQ ID NO | Type | Description |
|---|---|---|
| 1 | Protein | Human TDP-43 (UniProt Q13148-1, NP_031401.1) |
| 2 | DNA | Human TARDBP Coding Sequence (CCDS122.1) |
| 3 | DNA | Human TARDP cDNA (NM_007375.4) |
| 4 | Protein | Human TDP-43 Isoform 2 (UniProt Q13148-2) |
| 5 | Protein | Human TDP-43 PLD |
| 6 | Protein | Human TDP-43 PLD (28 AA) |
| 7 | Protein | Human hnRNPA2B1 (P22626-1, NP_112533.1) |
| 8 | DNA | Human HNRNPA2B1 CDS (CCDS43557.1) |
| 9 | DNA | Human HNRNPA2B1 cDNA (NM_031243.3) |
| 10 | Protein | Human hnRNPA2B1 v2 (P22626-2, NP_002128.1) |
| 11 | DNA | Human HNRNPA2B1 CDS v2 (CCDS5397.1) |
| 12 | DNA | Human HNRNPA2B1 cDNA v2 (NM_002137.4) |
| 13 | Protein | Human hnRNPA2B1 PLD |
| 14 | Protein | Human hnRNPA1 (P09651-1, NP_112420.1) |
| 15 | DNA | Human HNRNPA1 CDS (CCDS44909.1) |
| 16 | DNA | Human HNRNPA1 cDNA (NM_031157.4) |
| 17 | Protein | Human hnRNPA1 v2 (P09651-2, NP_002127.1) |
| 18 | DNA | Human HNRNPA1 CDS v2 (CCDS41793.1) |
| 19 | DNA | Human HNRNPA1 cDNA v2 (NM_002136.4) |
| 20 | Protein | Human hnRNPA1 v3 (P09651-3) |
| 21 | Protein | Human hnRNPA1 v2 PLD |
| 22 | Protein | PLD of Human TDP-43 PLDaromatic Variant |
| 23 | Protein | Human TDP-43 PLDaromatic Variant |
| 24 | Protein | Inserted Portion of PLD in Human TDP-43 PLD28aaVariant |
| 25 | Protein | PLD of Human TDP-43 PLD28aaVariant |
| 26 | Protein | Human TDP-43 PLD28aa Variant |
| 27 | Protein | PLD of Human TDP-43 PLDswap Variant |
| 28 | Protein | Human TDP-43 PLDswap Variant |
| 29-42 | DNA | Primers and Probes |
| 43 | Protein | Mouse TDP-43 (UniProt Q921F2-1; NP_663531.1) |
| 44 | DNA | Mouse Tardbp Coding Sequence (CCDS38971.1) |
| 45 | DNA | Mouse Tardp cDNA (NM_145556.4) |
| 46 | Protein | Mouse TDP-43 PLD |
| 47 | Protein | Mouse TDP-43 PLD (28 AA) |
| 48 | Protein | Mouse hnRNPA2B1 (UniProt O88569-1, NP_001361674.1, XP_006506436.2) |
| 49 | DNA | Mouse Hnrnpa2b1 Coding Sequence (CCDS90046.1) |
| 50 | DNA | Mouse Hnrnpa2b1 cDNA (XM_006506373.3) |
| 51 | DNA | Mouse Hnrnpa2b1 cDNA (NM_001374745.1) |
| 52 | Protein | Mouse hnRNPA2B1 v2 (UniProt O88569-2, NP_058086.2) |
| 53 | DNA | Mouse Hnrnpa2b1 v2 Coding Sequence (CCDS51774.1) |
| 54 | DNA | Mouse Hnrnpa2b1 v2 cDNA (NM_016806.3) |
| 55 | Protein | Mouse hnRNPA2B1 v3 (UniProt O88569-3; NP_872591.1) |
| 56 | DNA | Mouse Hnrnpa2b1 v3 Coding Sequence (CCDS51773.1) |
| 57 | DNA | Mouse Hnrnpa2b1 v3 cDNA (NM_182650.4) |
| 58 | Protein | Mouse hnRNPA2B1 PLD |
| 59 | Protein | Mouse hnRNPA1 (UniProt P49312-1; NP_034577.1) |
| 60 | DNA | Mouse Hnrnpa1 Coding Sequence (CCDS37233.1) |
| 61 | DNA | Mouse Hnrnpa1 cDNA (NM_010447.5) |
| 62 | Protein | Mouse hnRNPA1 v2 (UniProt P49312-2) |
| 63 | Protein | PLD of Mouse TDP-43 PLDaromatic Variant |
| 64 | Protein | Mouse TDP-43 PLDaromatic Variant |
| 65 | Protein | Inserted Portion of PLD in Mouse TDP-43 PLD28aaVariant |
| 66 | Protein | PLD of Mouse TDP-43 PLD28aaVariant |
| 67 | Protein | Mouse TDP-43 PLD28aa Variant |
| 68 | Protein | PLD of Mouse TDP-43 PLDswap Variant |
| 69 | Protein | Mouse TDP-43 PLDswap Variant |
| 70 | DNA | TDP-43 PLD swap coding sequence |
| 71 | DNA | TDP-43 28 amino acid swap coding sequence |
| 72 | DNA | TDP-43 aromatic variant coding sequence |
| 73 | Protein | Mouse hnRNPA1 PLD |

### EXAMPLES

### Example 1. Replacement of the Prion-Like Domain of TDP-43 with the PLD of hnRNPA2B1

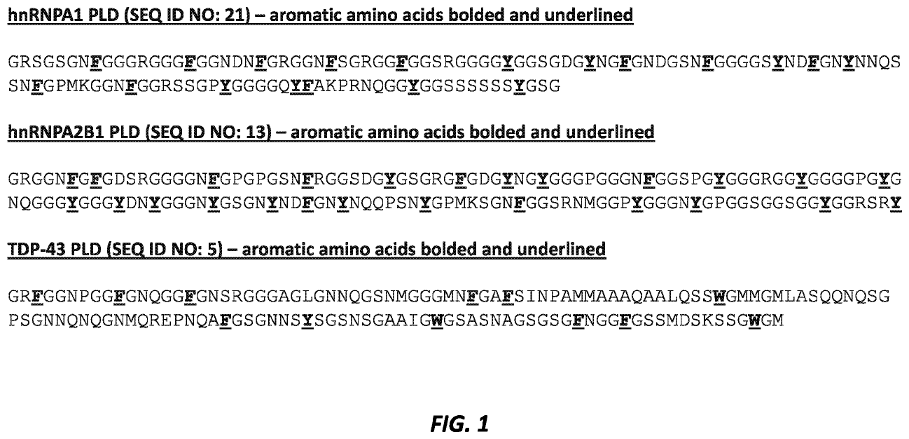

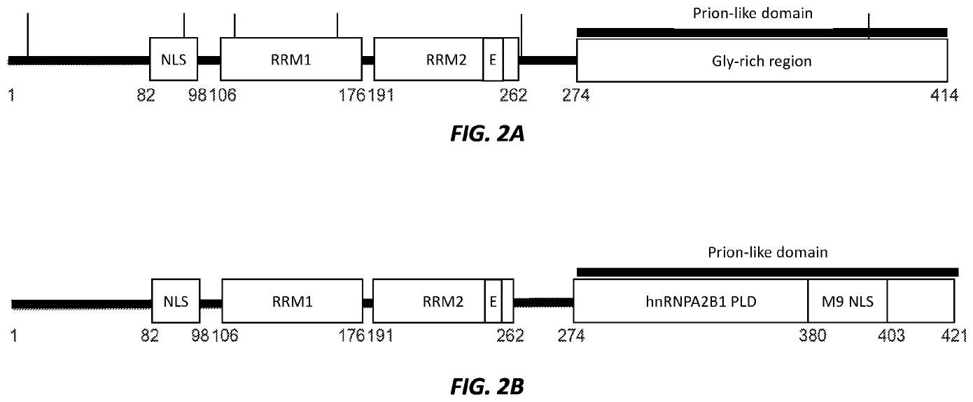

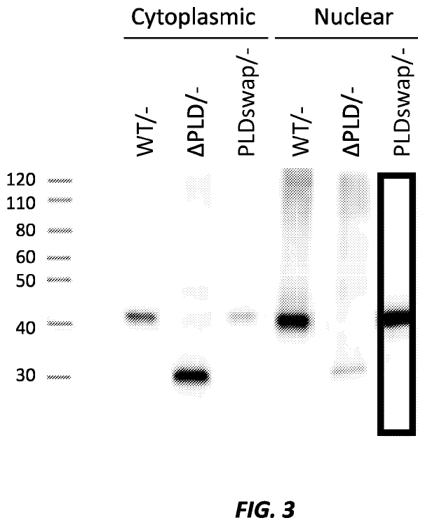

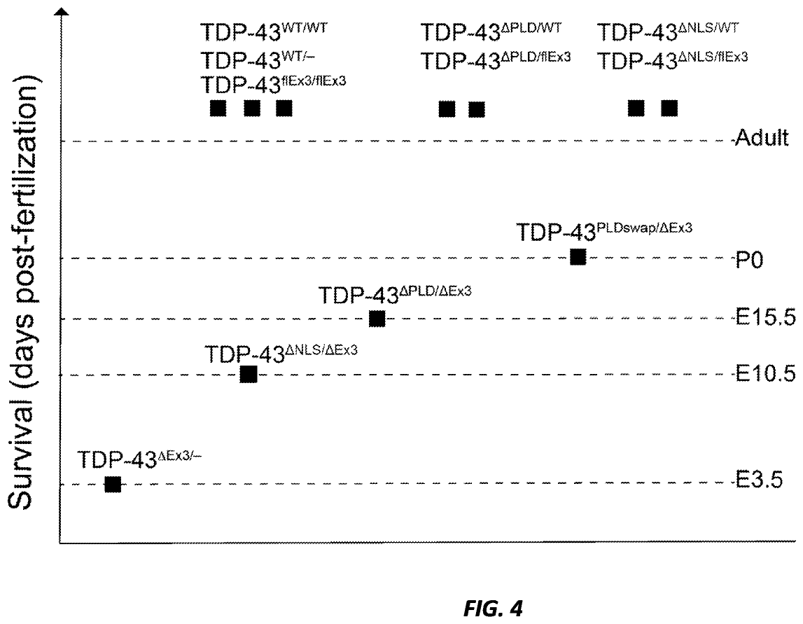

Several structural features of the TDP-43 protein have been identified, including a nuclear localization signal (NLS), two RNA recognition motifs (RRM1 and RRM2), a putative nuclear export signal (NES), and a large domain in the carboxyl-terminal half of the protein that has been described as a low complexity, poorly ordered, or prion-like domain (PLD). Of the mutations in TDP-43 that are associated with familial cases of ALS, most are found in the PLD. The PLD of TDP-43 has been heavily implicated in mediating the propensity of TDP-43 to form aggregates. Biochemical studies have shown that the even spacing of aromatic residues throughout PLDs allows efficient liquid-liquid phase separation but prevents the irreversible associations that lead to aggregation. The PLD of TDP-43 has fewer and less evenly spaced aromatic amino acids than other RNA binding proteins (FIG. 1) suggesting that re-organizing the spacing of aromatic residues throughout the PLD could prevent aggregation. To test this hypothesis, we created a PLDswap allele (amino acid sequence set forth in SEQ ID NO: 69; coding sequence set forth in SEQ ID NO: 70) that replaces the PLD of mouse TDP-43 with the PLD of mouse hnRNPA2B1 (FIG. 2B). The PLD of hnRNPA2B1 when compared to that of TDP-43 is comprised of evenly spaced aromatic residues (FIG. 1). Western blot analysis of subcellular fraction obtained from ES cell-derived motor neurons with the PLDswap allele as the only form of TDP-43 exhibited normal TDP-43 subcellular distribution (FIG. 3). To determine if this chimeric protein could substitute for TDP-43 we attempted to generate mice where this was the only form of TDP-43. We had previously confirmed that genetic ablation of TDP-43 results in embryonic lethality at e3.5-e5.5, and mice with a mutated NLS (ΔNLS) or a PLD deletion (ΔPLD) do not survive past embryonic day 12.5 and 15.5, respectively. Rather surprisingly, mice with the PLDswap allele as the only form of TDP-43 died at birth, suggesting that this form of TDP-43 can substitute for the function of TDP-43 during embryogenesis (FIG. 4).

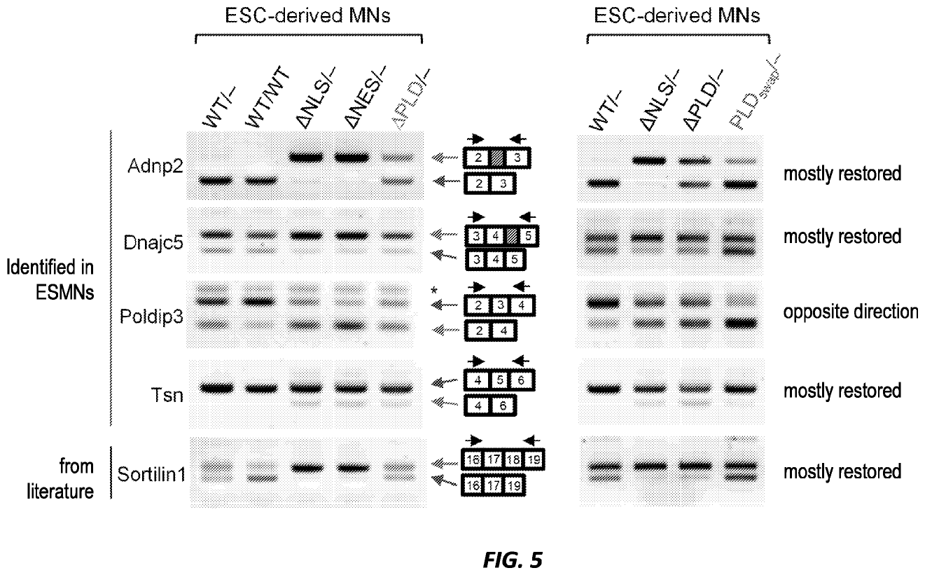

Lastly, we wanted to determine if this chimeric protein was able was able to retain the function of TDP-43. The most well-characterized function of TDP-43 is in regulating RNA splicing. Specifically, TDP-43 binds to intronic sequences to suppress cryptic exons from being aberrantly included in mRNA transcripts and can also control alternative splicing events. To assay for TDP-43 function, we carried out semi-quantitative RT-PCR for specific splicing events in Adnp2, Dnajc5, Poldip3, Tsn, and Sortilin1 determined to be TDP-43-dependent. We have previously shown that these events are disrupted by mutations that render the NLS non-functional (ΔNLS), deletion of the nuclear export signal (ΔNES) and deletion of the prion-like domain (ΔPLD). Surprisingly, in ES cell-derived motor neurons substitution of TDP-43's PLD with the PLD of hnRNPA2B1 TDP-43 splicing function is mostly retained (FIG. 5).

Together, these data demonstrate that the prion like domain of TDP-43 is amenable to engineering, suggesting that removing wild type TDP-43 and replacing it with a modified, aggregation-resistant form could prove to be of therapeutic value. It may also be that simply replacing the wild type protein would rescue phenotypes associated with the loss of functional TDP-43.

### Methods

Cell Culture. The ability of the chimeric TDP-43-hnRNP-A2B1 PLDswap, as the only form of the protein expressed by the cell, to support viability of embryonic stem (ES) cells and motor neurons derived from them (ESMNs) was tested by differentiation in culture. ES cells were cultured in embryonic stem cell medium (ESM; DMEM+15% fetal bovine serum+penicillin/streptomycin+glutamine+non-essential amino acids+nucleosides+β-mercaptoethanol+sodium pyruvate+LIF) for 2 days, during which the medium was changed daily. ES medium was replaced with 7 mL of ADFNK medium (advanced DMEM/F12+neurobasal medium+10% knockout serum+penicillin/streptomycin+glutamine+0-mercaptoethanol) 1 hour before trypsinization. ADFNK medium was aspirated, and ESCs were trypsinized with 0.05% trypsin-EDTA. Pelleted cells were resuspended in 12 mL of ADFNK and grown for two days in suspension. Cells were cultured for a further 4 days in ADFNK supplemented with retinoic acid (RA), smoothened agonist and purmorphamine to obtain limb-like motor neurons (ESMNs). Dissociated motor neurons were plated and matured in embryonic-stem-cell-derived motor neuron medium (ESMN; neurobasal medium+2% horse serum+B27+glutamine+penicillin/streptomycin+β-mercaptoethanol+10 ng/mL GDNF, BDNF, CNTF). The conditional knockout allele was activated using cre recombinase delivered via electroporation at the ES cell stage and ESMN stage.

Subcellular Fractionation of TDP-43. The subcellular localization of the chimeric TDP-43-PLDswap protein was analyzed using an antibody that recognizes the N-terminus of the TDP-43 polypeptide (α-TDP-43 N-term) and an antibody that recognizes the C-terminal prion like domain of the TDP-43 polypeptide (α-TDP-43 C-term) (Proteintech, Rosemont, IL). Soluble cytoplasmic protein extracts were prepared by incubating ES cell-derived MNs in ice-cold lysis buffer (10 mM KCl, 10 mM Tris-HCl, pH 7.4, 1 mM MgCl2, 1 mM DTT, 0.01% NP-40) supplemented with protease and phosphatase inhibitors (Roche) for 10 minutes on ice. Cells were then passed through a 27-gauge syringe five times. Following centrifugation at 4° C. for 5 minutes at 4000 rpm, the protein supernatant that comprises the soluble cytoplasmic extract was collected. Insoluble nuclear protein extracts were prepared by resuspending the pellet in an equal volume of RBS-100 buffer (10 mM Tris-HCl pH 7.4, 2.5 mM MgCl2, 100 mM NaCl, 0.1% NP-40) supplemented with protease and phosphatase. Equal volumes of 2×SDS sample buffer was added to each fraction and samples were heated to 90° C. Equal volumes of each fraction were then loaded onto a 10% SDS gel and electrophoresed for 50 minutes at 225V followed by western blotting for TDP-43 using the α-TDP-43-N-term antibody or the α-TDP-43-C-term antibody, the latter of which would not recognize the PLDswap mutant.

Generation of Mice Expressing the Chimeric TDP-43-hnRNPA2B1 ‘PLDswap’ Protein. Although deletion of TDP-43 results in embryonic lethality, embryonic stem cells expressing only a mutant ΔNLS TDP-43 gene or a mutant ΔPLD TDP-43 gene from the endogenous TARDBP locus are viable and may be differentiated into motor neurons in vitro. These data raise the possibility that embryonic stem cells expressing a mutant TDP-43 polypeptide lacking a functional structural domain from an endogenous TARDBP locus may be viable and useful in creating animal models of TDP-43 proteinopathies. For example, such embryonic stem cells may be used to generate non-human animals, e.g., mice, expressing mutant TDP-43 proteins lacking a functional structural domain to examine the role of TDP-43 structural domains in normal and pathological biological processes.

To create embryos or animals that express a chimeric TDP-43-hnRNPA2B1 protein, the VELOCIMOUSE® method (Dechiara (2009) Methods Mol. Biol. 530:311-324 and Poueymirou et al. (2007) Nat. Biotechnol. 25:91-99, each of which is herein incorporated by reference in its entirety for all purposes) was used. Targeted ES cells comprising (i) at an endogenous TARDBP locus, a TARDBP gene comprising a chimeric TDP-43-hnRNPA2B1 ‘PLDswap’ allele and (ii) a null TDP-43 allele produced by upon Cre-mediated deletion of the floxed exon 3 (−) were injected into uncompacted 8-cell stage Swiss Webster embryos. The viability of embryos after fertilization was examined and the ability to produce live-born F0 generation mice was assessed.

RT-PCR of Splicing Events. Total RNA was isolated from ES-cell derived motor neurons using Trizol reagent followed by DNase treatment. For cDNA, 1 μg of total RNA was used as a template for cDNA synthesis using SuperScript IV First-Strand Synthesis System (ThermoFisher cat #18091050). Reactions were carried out in a volume of 20 μL and then brought up to a final volume of 100 μL after cDNA synthesis was complete. PCR reactions were carried out using Q5 2× MasterMix (NEB) with 2 μL of cDNA template, 1 mM of each forward and reverse primer, in a total reaction volume of 25 μL. For each transcript, PCRs were first optimized to determine the cycle number that allowed for amplification that remained unsaturated and within a linear range. Reactions were run on 1.8% agarose gels in 1×TAE and bands were visualized using SybrSafe. The primers used and corresponding cycle numbers are listed below.

TABLE 4
Primers and Corresponding Cycle Numbers.

| Target | Forward primer (5′ to 3′) | Reverse primer (5′ to 3′) | Cycles |
|---|---|---|---|
| Sortilin1 E16/19 | GCATGAGTTAGAGTTCTGTCTG | CTTCCGCCACAGACATATTTC | 30 |
| | (SEQ ID NO: 29) | (SEQ ID NO: 30) | |
| Dnajc5 E3/5 | CTACTTCGTACTCTCCAGCTG | GATGCTGGCTGTATGACGATC | 29 |
| | (SEQ ID NO: 31) | (SEQ ID NO: 32) | |
| Poldip3 E2/4 | GAGAAGATCAGCTTGAAGAGG | CACCACAATGTCATCATCTTC | 30 |
| | (SEQ ID NO: 33) | (SEQ ID NO: 34) | |
| Adnp2 E2/3 | GCAGAATCTTGACAACATCAGG | GCTTTCTTTCCAGAAGGTTCC | 29 |
| | (SEQ ID NO: 35) | (SEQ ID NO: 36) | |
| Caskin1 E14/17 | CACCGAAGAAGCTGGAATC | CCAGGTGATATCGGTGATG | 30 |
| | (SEQ ID NO: 37) | (SEQ ID NO: 38) | |
| Tsn E4/6 | GTTTCATGAGCATTGGCGGTTC | GTAGTCTCCAGCAGTGACACTG | 30 |
| | (SEQ ID NO: 39) | (SEQ ID NO: 40) | |
| GAPDH E3/5 | ACCACCATGGAGAAGGCCGGG | CAGTGATGGCATGGACTGTGG | 26 |
| | (SEQ ID NO: 41) | (SEQ ID NO: 42) | |

### Example 2. Function of TDP-43 PLDswap in Neonatal Mice

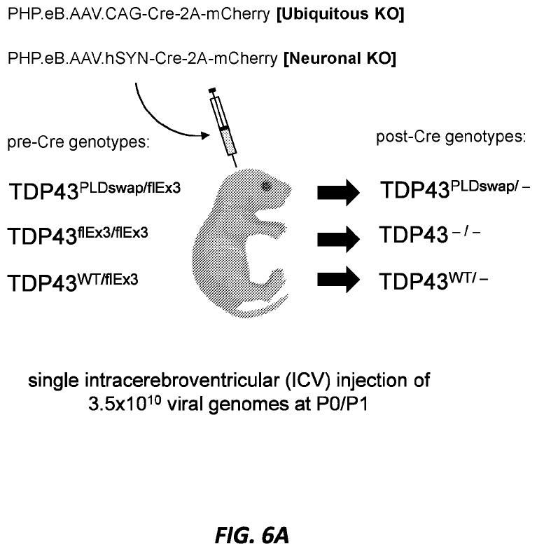

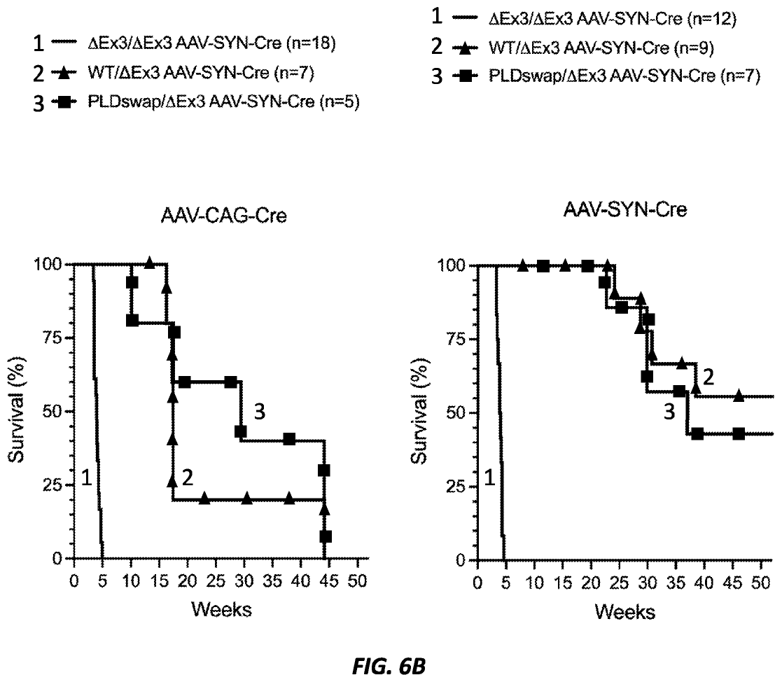

The requirement for TDP-43 function during embryogenesis and fetal development is essential and may encompass functions of TDP-43 that are not relevant to the survival of post-natal neurons in vivo. Therefore, to further characterize the functionality of the PLDswap form of TDP-43 while also circumventing any requirement for TDP-43 prenatally, we carried out a set of experiments to test whether the PLDswap form of TDP-43 could compensate for wild-type TDP-43 in a post-natal mouse. To do so, we utilized mice that harbor an exon 3 floxed conditional knockout (cKO) allele (“flEx3”) that undergoes Cre-mediated recombination to produce a ΔEx3 knockout allele when in the presence of Cre. We then paired this conditional allele with the PLDswap mutant (TDP-43^{flEx3/PLDswap}) to allow for conditional removal of WT TDP-43, leaving PLDswap as the only form of the protein in Cre-expressing cells. As negative and positive controls, respectively, we used mice that are homozygous for the flEx3 conditional allele (TDP-43^{flEx3/flEx3}) and mice that have one WT allele paired with the conditional allele (TDP-43^{flEx3/WT}) To induce conditional knockout of the floxed WT allele, a PHP.eB.AAV virus expressing a Cre-2A-mCherry cassette under the control of either the ubiquitous CAG promoter (PHP.eB.AAV-CAG-Cre-2A-mCherry) or the neuron-specific human synapsin promoter (PHP.eB.AAV-hSYN-Cre-2A-mCherry) was injected by intracerebroventricular (i.e.v.) injection into P0 newborn mouse pups of all three genotypes. See FIG. 6A. Given the essential requirement of TDP-43, we used survival as a readout of TDP-43 function in these animals. As expected due to the essentiality of TDP-43, mice that were homozygous for the cKO allele (TDP-43^{ΔEx/ΔEX3}) did not survive past 5 weeks after Cre delivery in both the ubiquitous (CAG) or neuron-specific (SYN) context. In contrast, both TDP-43^{flEx3/WT} and TDP-43^{flEx3/PLDswap} mice survived significantly longer and to a remarkably similar extent, suggesting that the PLDswap form of TDP-43 is able to function long-term as a replacement for wild-type TDP-43 protein in neurons. See FIG. 6B.

One caveat of this experimental system is that high expression of Cre recombinase in neurons can display toxicity, which likely accounts for the shorter than expected survival of our positive control TDP-43^{flEx3/WT} mice, especially in the CAG-Cre context where expression is ultra-high. Therefore, this system may be underestimating the extent to which the PLDswap form of TDP-43 can compensate for TDP-43 function in vivo.

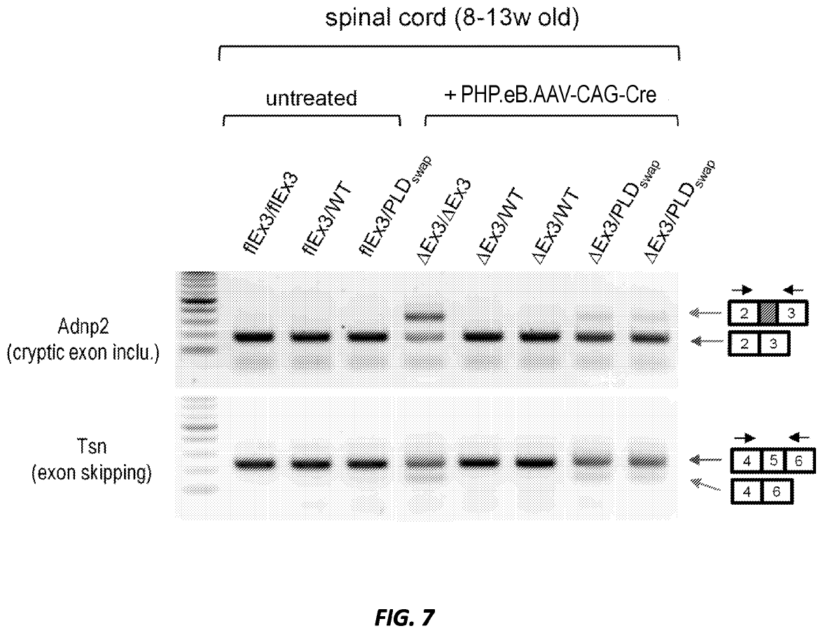

To support the survival data, we also directly tested the ability of the PLDswap protein to function in TDP-43-dependent splicing events. To do so, we collected spinal cord tissue from mice with or without Cre-mediated removal of the conditional wild-type TDP-43 allele and carried out semi-quantitative RT-PCR for specific TDP-43-dependent splicing events. We tested two mRNAs known to undergo TDP-43 splicing: Adnp2 mRNA and Tsn mRNA. While all animals without CAG-Cre expression displayed normal transcript processing, homozygous removal of the conditional allele (and TDP-43^{ΔEx3/ΔEx3}) triggered the mis-splicing of both transcripts (i.e., cryptic exon inclusion in Adnp2 and exon 5 skipping in Tsn). In comparison, both transcripts were spliced normally in Cre-treated TDP-43^{ΔEx3/WT} mice. In the Cre-treated TDP-43^{flEx3/PLDswap} mice, there was a trend towards rescue of the splicing defects, especially in the cryptic exon inclusion in Adnp2. See FIG. 7. This analysis of two genes that are dependent on TDP-43 for their normal splicing offers molecular support to the notion that the chimeric PLDswap form of TDP-43 has the capacity to function like normal TDP-43 to an extent.

### Example 3. Replacement of a Portion the Prion-Like Domain of TDP-43 with a Portion of the PLD of hnRNPA2B1

As explained in Example 1, the PLD of TDP-43 has fewer and less evenly spaced aromatic amino acids than other RNA binding proteins (FIG. 1) suggesting that re-organizing the spacing of aromatic residues throughout the PLD could prevent aggregation. To test this hypothesis, we created a PLD28aa allele (amino acid sequence set forth in SEQ TD NO: 67; coding sequence set forth in SEQ ID NO: 71) that replaces a highly conserved 28 amino acid stretch within the PLD of mouse TDP-43, shown to be important for aggregation in yeast, with a sequence from the PLD of mouse hnRNPA2B1. The PLD of hnRNPA2B1 when compared to that of TDP-43 is comprised of evenly spaced aromatic residues (FIG. 1). The PLD28aa allele was confirmed to be viable as the only form of TDP-43 in mouse embryonic stem cells (data not shown). As in Example 1, western blot analysis of subcellular fraction obtained from ES cell-derived motor neurons with the PLD28aa allele as the only form of TDP-43 is performed to analyze TDP-43 subcellular distribution. As in Example 1, to determine if this chimeric protein could substitute for TDP-43, mice are generated where this was the only form of TDP-43. Embryonic development is assessed in the mice. Experiments are also performed in Example 1 to determine if this chimeric protein is able was able to retain the function of TDP-43, specifically by assessing TDP-43 splicing function in ES cell-derived motor neurons.

### Example 4. Introduction of Uniformly Spaced Aromatic Residues (“Stickers”) within the Prion-Like Domain of TDP-43 to Promote Liquid-Liquid Phase Separation (LLPS) but Inhibit Aggregation

As explained in Example 1, the PLD of TDP-43 has fewer and less evenly spaced aromatic amino acids than other RNA binding proteins (FIG. 1) suggesting that re-organizing the spacing of aromatic residues throughout the PLD could prevent aggregation. To test this hypothesis, we created a PLDaromatic allele (amino acid sequence set forth in SEQ ID NO: 64; coding sequence set forth in SEQ ID NO: 72) that introduces uniformly spaced aromatic residues (“stickers”) within the prion-like domain of TDP-43 to promote liquid-liquid phase separation (LLPS) but inhibit aggregation. The PLDaromatic allele was confirmed to be viable as the only form of TDP-43 in mouse embryonic stem cells (data not shown). As in Example 1, western blot analysis of subcellular fraction obtained from ES cell-derived motor neurons with the PLDaromatic allele as the only form of TDP-43 is performed to analyze TDP-43 subcellular distribution. As in Example 1, to determine if this chimeric protein could substitute for TDP-43, mice are generated where this was the only form of TDP-43. Embryonic development is assessed in the mice. Experiments are also performed in Example 1 to determine if this chimeric protein is able was able to retain the function of TDP-43, specifically by assessing TDP-43 splicing function in ES cell-derived motor neurons.

We claim:

1. A TAR DNA-binding protein 43 (TDP-43) variant in which a prion-like domain (PLD) of the TDP-43 variant is mutated to have more aromatic amino acids and/or aromatic amino acids that are more evenly spaced than in a PLD from a wild type TDP-43.

2. The TDP-43 variant of claim 1, wherein the PLD is mutated to have more aromatic amino acids.

3. The TDP-43 variant of claim 1 or 2, wherein the PLD is mutated to have aromatic amino acids that are more evenly spaced than in the PLD from the wild type TDP-43.

4. The TDP-43 variant of any one of claims 1-3, wherein the PLD is mutated to have more aromatic amino acids and aromatic amino acids that are more evenly spaced than in the PLD from the wild type TDP-43.

5. The TDP-43 variant of any one of claims 1-4, wherein the PLD in the TDP-43 variant is at least about 90%, at least about 95%, at least about 96%, at least about 97%, at least about 98%, at least about 99%, or 100% identical to the sequence set forth in SEQ ID NO: 22, or
wherein the PLD in the TDP-43 variant is at least about 90%, at least about 95%, at least about 96%, at least about 97%, at least about 98%, at least about 99%, or 100% identical to the sequence set forth in SEQ ID NO: 63.

6. The TDP-43 variant of any one of claims 1-5, wherein the PLD in the TDP-43 variant comprises, consists essentially of, or consists of the sequence set forth in SEQ ID NO: 22, or
wherein the PLD in the TDP-43 variant comprises, consists essentially of, or consists of the sequence set forth in SEQ ID NO: 63.

7. The TDP-43 variant of any one of claims 1-6, wherein the TDP-43 variant is at least about 90%, at least about 95%, at least about 96%, at least about 97%, at least about 98%, at least about 99%, or 100% identical to the sequence set forth in SEQ ID NO: 23, or
wherein the TDP-43 variant is at least about 90%, at least about 95%, at least about 96%, at least about 97%, at least about 98%, at least about 99%, or 100% identical to the sequence set forth in SEQ ID NO: 64.

8. The TDP-43 variant of any one of claims 1-7, wherein the TDP-43 variant comprises, consists essentially of, or consists of the sequence set forth in SEQ ID NO: 23, or
wherein the TDP-43 variant comprises, consists essentially of, or consists of the sequence set forth in SEQ ID NO: 64.

9. The TDP-43 variant of any one of claims 1-4, wherein a portion of the PLD from the wild type TDP-43 is replaced with at least a portion of a PLD from a different RNA-binding protein in the variant TDP-43, optionally wherein:
(i) the portion of the PLD from the wild type TDP-43 that is replaced is at least about 10, at least about 15, at least about 20, at least about 25, or at least about 28 amino acids;
(ii) the portion of the PLD from the wild type TDP-43 that is replaced is between about 10 and about 50, between about 20 and about 40, or between about 25 and about amino acids;
(iii) the portion of the PLD that is replaced is about 28 amino acids; or
(iv) the portion of the PLD that is replaced comprises, consists essentially of, or consists of SEQ ID NO: 6, or the portion of the PLD that is replaced comprises, consists essentially of, or consists of SEQ ID NO: 47.

10. The TDP-43 variant of claim 9, wherein the different RNA-binding protein is hnRNPA2B1.

11. The TDP-43 variant of claim 10, wherein the portion of the PLD from hnRNPA2B1 is at least about 90%, at least about 95%, at least about 96%, at least about 97%, at least about 98%, at least about 99%, or 100% identical to the sequence set forth in SEQ ID NO: 24, or
wherein the portion of the PLD from hnRNPA2B1 is at least about 90%, at least about 95%, at least about 96%, at least about 97%, at least about 98%, at least about 99%, or 100% identical to the sequence set forth in SEQ ID NO: 65.

12. The TDP-43 variant of claim 10 or 11, wherein the portion of the PLD from hnRNPA2B1 comprises, consists essentially of, or consists of the sequence set forth in SEQ ID NO: 24, or
wherein the portion of the PLD from hnRNPA2B1 comprises, consists essentially of, or consists of the sequence set forth in SEQ ID NO: 65.

13. The TDP-43 variant of any one of claims 10-12, wherein the PLD in the TDP-43 variant is at least about 90%, at least about 95%, at least about 96%, at least about 97%, at least about 98%, at least about 99%, or 100% identical to the sequence set forth in SEQ ID NO: 25, or
wherein the PLD in the TDP-43 variant is at least about 90%, at least about 95%, at least about 96%, at least about 97%, at least about 98%, at least about 99%, or 100% identical to the sequence set forth in SEQ ID NO: 66.

14. The TDP-43 variant of any one of claims 10-13, wherein the PLD in the TDP-43 variant comprises, consists essentially of, or consists of the sequence set forth in SEQ ID NO: 25, or
wherein the PLD in the TDP-43 variant comprises, consists essentially of, or consists of the sequence set forth in SEQ ID NO: 66.

15. The TDP-43 variant of any one of claims 10-14, wherein the TDP-43 variant is at least about 90%, at least about 95%, at least about 96%, at least about 97%, at least about 98%, at least about 99%, or 100% identical to the sequence set forth in SEQ ID NO: 26, or
wherein the TDP-43 variant is at least about 90%, at least about 95%, at least about 96%, at least about 97%, at least about 98%, at least about 99%, or 100% identical to the sequence set forth in SEQ ID NO: 67.

16. The TDP-43 variant of any one of claims 10-15, wherein the TDP-43 variant comprises, consists essentially of, or consists of the sequence set forth in SEQ ID NO: 26, or
wherein the TDP-43 variant comprises, consists essentially of, or consists of the sequence set forth in SEQ ID NO: 67.

17. The TDP-43 variant of any one of claims 1-4, wherein the PLD from the wild type TDP-43 is replaced with a PLD from a different RNA-binding protein in the variant TDP-43.

18. The TDP-43 variant of claim 17, wherein the different RNA-binding protein is hnRNPA2B1.

19. The TDP-43 variant of claim 18, wherein the PLD in the TDP-43 variant is at least about 90%, at least about 95%, at least about 96%, at least about 97%, at least about 98%, at least about 99%, or 100% identical to the sequence set forth in SEQ ID NO: 27 or 13, or
wherein the PLD in the TDP-43 variant is at least about 90%, at least about 95%, at least about 96%, at least about 97%, at least about 98%, at least about 99%, or 100% identical to the sequence set forth in SEQ ID NO: 68 or 58.

20. The TDP-43 variant of claim 18 or 19, wherein the PLD in the TDP-43 variant comprises, consists essentially of, or consists of the sequence set forth in SEQ ID NO: 27 or 13, or
wherein the PLD in the TDP-43 variant comprises, consists essentially of, or consists of the sequence set forth in SEQ ID NO: 68 or 58.

21. The TDP-43 variant of any one of claims 18-20, wherein the TDP-43 variant is at least about 90%, at least about 95%, at least about 96%, at least about 97%, at least about 98%, at least about 99%, or 100% identical to the sequence set forth in SEQ ID NO: 28, or
wherein the TDP-43 variant is at least about 90%, at least about 95%, at least about 96%, at least about 97%, at least about 98%, at least about 99%, or 100% identical to the sequence set forth in SEQ ID NO: 69.

22. The TDP-43 variant of any one of claims 18-21, wherein the TDP-43 variant comprises, consists essentially of, or consists of the sequence set forth in SEQ ID NO: 28, or
wherein the TDP-43 variant comprises, consists essentially of, or consists of the sequence set forth in SEQ ID NO: 69.

23. The TDP-43 variant of any one of claims 1-22, wherein the TDP-43 variant is less prone to aggregation than the wild type TDP-43.

24. The TDP-43 variant of any one of claims 1-23, wherein the TDP-43 variant retains functions of wild type TDP-43 in splicing regulation.

25. The TDP-43 variant of any one of claims 1-24, wherein the TDP-43 variant is predominantly nuclear and/or retains the subcellular distribution of wild type TDP-43.

26. The TDP-43 variant of any one of claims 1-25, wherein the TDP-43 variant retains functions of wild type TDP-43 during embryonic development.

27. The TDP-43 variant of any one of claims 1-26, wherein the TDP-43 variant is a human TDP-43 variant.

28. The TDP-43 variant of any one of claims 1-27 for use in the treatment of a TDP-43 proteinopathy in a subject, optionally wherein the TDP-43 proteinopathy is amyotrophic lateral sclerosis (ALS).

29. The TDP-43 variant of any one of claims 1-27 for use in the prevention of a TDP-43 proteinopathy in a subject, optionally wherein the TDP-43 proteinopathy is amyotrophic lateral sclerosis (ALS).

30. Use of the TDP-43 variant of any one of claims 1-27 for the manufacture of a medicament for the treatment of a TDP-43 proteinopathy, optionally wherein the TDP-43 proteinopathy is amyotrophic lateral sclerosis (ALS).

31. Use of the TDP-43 variant of any one of claims 1-27 for the manufacture of a medicament for the prevention of a TDP-43 proteinopathy, optionally wherein the TDP-43 proteinopathy is amyotrophic lateral sclerosis (ALS).

32. A nucleic acid encoding the TDP-43 variant of any one of claims 1-27.

33. The nucleic acid of claim 32, wherein the nucleic acid is a messenger RNA.

34. The nucleic acid of claim 32, wherein the nucleic acid comprises DNA, optionally wherein the DNA comprises a complementary DNA (cDNA).

35. The nucleic acid of claim 34, wherein the nucleic acid is in an expression construct comprising a promoter operably linked to the nucleic acid encoding the TDP-43 variant, optionally wherein the promoter is a neuron-specific promoter or a constitutive promoter.

36. The nucleic acid of claim 35, wherein the promoter is a constitutive promoter, a tissue-specific promoter, or an inducible promoter.

37. The nucleic acid of claim 36, wherein the promoter is a neuron-specific promoter, optionally wherein the promoter is a synapsin-1 promoter, and optionally wherein the promoter is a human synapsin-1 promoter.

38. The nucleic acid of any one of claims 34-37, wherein the nucleic acid is in a vector.

39. The nucleic acid of claim 38, wherein the vector is a viral vector.

40. The nucleic acid of claim 38, wherein the viral vector is a lentivirus vector or an adeno-associated virus (AAV) vector.

41. The nucleic acid of claim 40, wherein the vector is the AAV vector, optionally wherein the AAV vector is an AAV-PHP.eB vector.

42. The nucleic acid of any one of claims 32-41, wherein the nucleic acid is codon-optimized for expression in human cells.

43. A cell comprising:
(i) the TDP-43 variant of any one of any one of claims 1-27; or
(ii) the nucleic acid of any one of claims 32-42, wherein the TDP-43 variant is expressed.

44. The cell of claim 43, wherein the cell is a mammalian cell.

45. The cell of claim 44, wherein the mammalian cell is a human cell, a rodent cell, a mouse cell, or a rat cell.

46. The cell of claim 45, wherein the cell is the human cell.

47. The cell of any one of claims 43-46, wherein the cell is a neuron, a glial cell, or a muscle cell.

48. The cell of any one of claims 43-47, wherein the cell is in vivo in a subject.

49. The cell of claim 48, wherein the cell is a neuron in the brain of the subject.

50. The cell of any one of claims 43-47, wherein endogenous TDP-43 is not expressed in the cell.

51. The cell of claim 50, wherein the endogenous TARDBP genomic locus comprises a mutation that prevents expression of endogenous TDP-43 in the cell.

52. The cell of any one of claims 43-51, further comprising an agent that reduces or eliminates expression of endogenous TDP-43 in the cell.

53. The cell of claim 52, wherein the agent comprises an antisense oligonucleotide or an RNAi agent targeting endogenous TARDBP messenger RNA or a nucleic acid encoding the antisense oligonucleotide or the RNAi agent.

54. The cell of claim 52, wherein the agent comprises a nuclease agent targeting the endogenous TARDBP genomic locus or one or more nucleic acids encoding the nuclease agent.

55. The cell of claim 54, wherein the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA.

56. The cell of claim 55, wherein the nuclease agent is the Cas protein and the guide RNA, optionally wherein the Cas protein is a Cas9 protein.

57. The cell of any one of claim 43-56, wherein the cell has a genetically modified endogenous TARDBP genomic locus, wherein the nucleic acid is integrated at the endogenous TARDBP genomic locus.

58. The cell of claim 57, wherein the cell is heterozygous for the integrated nucleic acid.

59. The cell of claim 57, wherein the cell is homozygous for the integrated nucleic acid.

60. The cell of any one of claims 57-59, wherein the nucleic acid is operably linked to the endogenous TARDBP promoter.

61. The cell of any one of claims 57-60, wherein the TDP-43 variant is expressed from the endogenous TARDBP genomic locus and replaces expression of the endogenous TDP-43.

62. The cell of any one of claims 43-61, wherein the cell has reduced TDP-43 aggregation compared to a control cell without the TDP-43 variant or the nucleic acid.

63. A non-human animal comprising:
(i) the TDP-43 variant of any one of any one of claims 1-27; or
(ii) the nucleic acid of any one of claims 32-42, wherein the TDP-43 variant is expressed.

64. The non-human animal of claim 63, wherein the non-human animal is a mammal.

65. The non-human animal of claim 64, wherein the non-human animal is a rodent, a mouse, or a rat.

66. The non-human animal of claim 65, wherein the non-human animal is the mouse.

67. The non-human animal of any one of claims 63-66, wherein the TDP-43 variant or the nucleic acid is in a neuron, a glial cell, or a muscle cell in the non-human animal.

68. The non-human animal of any one of claims 63-67, wherein the TDP-43 variant or the nucleic acid is in the neuron.

69. The non-human animal of claim 68, wherein the neuron is in the brain of the non-human animal.

70. The non-human animal of any one of claims 63-67, wherein endogenous TDP-43 is not expressed in the non-human animal.

71. The non-human animal of claim 70, wherein the endogenous TARDBP genomic locus comprises a mutation that prevents expression of endogenous TDP-43 in the non-human animal.

72. The non-human animal of any one of claims 63-71, further comprising an agent that reduces or eliminates expression of endogenous TDP-43 in the non-human animal.

73. The non-human animal of claim 72, wherein the agent comprises an antisense oligonucleotide or an RNAi agent targeting endogenous TARDBP messenger RNA or a nucleic acid encoding the antisense oligonucleotide or the RNAi agent.

74. The non-human animal of claim 72, wherein the agent comprises a nuclease agent targeting the endogenous TARDBP genomic locus or one or more nucleic acids encoding the nuclease agent.

75. The non-human animal of claim 74, wherein the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA.

76. The non-human animal of claim 75, wherein the nuclease agent is the Cas protein and the guide RNA, optionally wherein the Cas protein is a Cas9 protein.

77. The non-human animal of any one of claim 63-76, wherein the non-human animal has a genetically modified endogenous TARDBP genomic locus, wherein the nucleic acid is integrated at the endogenous TARDBP genomic locus.

78. The non-human animal of claim 77, wherein the non-human animal is heterozygous for the integrated nucleic acid.

79. The non-human animal of claim 77, wherein the non-human animal is homozygous for the integrated nucleic acid.

80. The non-human animal of any one of claims 77-79, wherein the nucleic acid is operably linked to the endogenous TARDBP promoter.

81. The non-human animal of any one of claims 77-80, wherein the TDP-43 variant is expressed from the endogenous TARDBP genomic locus and replaces expression of the endogenous TDP-43.

82. The non-human animal of any one of claims 63-81, wherein the non-human animal has reduced TDP-43 aggregation compared to a control non-human animal without the TDP-43 variant or the nucleic acid.

83. A method of making the non-human animal of any one of claims 63-83, comprising administering the TDP-43 variant or the nucleic acid to the non-human animal.

84. A method of making the non-human animal of any one of claims 77-82, comprising:
(I)(a) modifying the genome of a pluripotent non-human animal cell to comprise the genetically modified endogenous TARDBP genomic locus;
(b) identifying or selecting the genetically modified pluripotent non-human animal cell comprising the genetically modified endogenous TARDBP genomic locus;
(c) introducing the genetically modified pluripotent non-human animal cell into a non-human animal host embryo; and
(d) gestating the non-human animal host embryo in a surrogate mother; or
(II)(a) modifying the genome of a non-human animal one-cell stage embryo to comprise the genetically modified endogenous TARDBP genomic locus;
(b) selecting the genetically modified non-human animal one-cell stage embryo comprising the genetically modified endogenous TARDBP genomic locus; and
(c) gestating the genetically modified non-human animal one-cell stage embryo in a surrogate mother.

85. A method comprising administering to a cell:
(i) the TDP-43 variant of any one of any one of claims 1-27; or
(ii) the nucleic acid of any one of claims 32-42, wherein the TDP-43 variant is expressed.

86. The method of claim 85, wherein the cell is a mammalian cell.

87. The method of claim 86, wherein the mammalian cell is a human cell, a rodent cell, a mouse cell, or a rat cell.

88. The method of claim 87, wherein the cell is the human cell.

89. The method of any one of claims 85-88, wherein the cell is a neuron, a glial cell, or a muscle cell.

90. The method of any one of claims 85-89 wherein the cell is in vivo in a subject.

91. The method of claim 90, wherein the cell is a neuron in the brain of the subject.

92. The method of claim 90 or 91, wherein the TDP-43 variant or the nucleic acid is administered to the subject via intracerebroventricular injection, intracranial injection, or intrathecal injection.

93. The method of any one of claims 85-92, further comprising administering to the cell an agent that reduces or eliminates expression of endogenous TDP-43 in the cell.

94. The method of claim 93, wherein the agent comprises an antisense oligonucleotide or an RNAi agent targeting endogenous TARDBP messenger RNA or a nucleic acid encoding the antisense oligonucleotide or the RNAi agent.

95. The method of claim 93, wherein the agent comprises a nuclease agent targeting the endogenous TARDBP genomic locus or one or more nucleic acids encoding the nuclease agent.

96. The method of claim 95, wherein the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA.

97. The method of claim 96, wherein the nuclease agent is the Cas protein and the guide RNA, optionally wherein the Cas protein is a Cas9 protein.

98. The method of any one of claims 95-97, wherein the method comprises administering the nucleic acid encoding the TDP-43 variant, wherein the nuclease agent cleaves the endogenous TARDBP genomic locus, the nucleic acid encoding the TDP-43 variant is inserted at or recombines with the cleaved endogenous TARDBP genomic locus, wherein the TDP-43 variant is expressed from the endogenous TARDBP genomic locus and replaces expression of the endogenous TDP-43.

99. The method of any one of claims 85-98, wherein endogenous TDP-43 in the cell is prone to aggregation, and wherein the method reduces TDP-43 aggregation in the cell.

100. The method of any one of claims 85-99, wherein there is aberrant splicing regulation by endogenous TDP-43 in the cell, and wherein the TDP-43 variant rescues aberrant TDP-43 splicing regulation in the cell.

101. The method of any one of claims 85-100, wherein there is aberrant subcellular distribution of endogenous TDP-43 in the cell, and wherein the TDP-43 variant rescues aberrant subcellular distribution of endogenous TDP-43 in the cell.

102. A method of treating a TDP-43 proteinopathy in a subject, comprising administering to one or more cells in the subject:
(i) the TDP-43 variant of any one of any one of claims 1-27; or
(ii) the nucleic acid of any one of claims 32-42, wherein the TDP-43 variant is expressed in the one or more cells in the subject.

103. A method of preventing a TDP-43 proteinopathy in a subject, comprising administering to one or more cells in the subject:
(i) the TDP-43 variant of any one of any one of claims 1-27; or
(ii) the nucleic acid of any one of claims 32-42, wherein the TDP-43 variant is expressed in the one or more cells in the subject.

104. The method of claim 102 or 103, wherein the TDP-43 proteinopathy is amyotrophic lateral sclerosis (ALS).

105. The method of any one of claims 102-104, wherein the subject is a mammal.

106. The method of any one of claims 102-105, wherein the subject is a human.

107. The method of any one of claims 102-106, wherein the one or more cells comprise neurons in the brain of the subject.

108. The method of any one of claims 102-107, wherein the TDP-43 variant or the nucleic acid is administered to the subject via intracerebroventricular injection, intracranial injection, or intrathecal injection.

109. The method of any one of claims 102-108, further comprising administering to the one or more cells an agent that reduces or eliminates expression of endogenous TDP-43 in the one or more cells.

110. The method of claim 109, wherein the agent comprises an antisense oligonucleotide or an RNAi agent targeting endogenous TARDBP messenger RNA or a nucleic acid encoding the antisense oligonucleotide or the RNAi agent.

111. The method of claim 109, wherein the agent comprises a nuclease agent targeting the endogenous TARDBP genomic locus or one or more nucleic acids encoding the nuclease agent.

112. The method of claim 111, wherein the nuclease agent is a Zinc Finger Nuclease (ZFN), a Transcription Activator-Like Effector Nuclease (TALEN), or a Clustered Regularly Interspaced Short Palindromic Repeats (CRISPR)-associated (Cas) protein and a guide RNA.

113. The method of claim 112, wherein the nuclease agent is the Cas protein and the guide RNA, optionally wherein the Cas protein is a Cas9 protein.

114. The method of any one of claims 111-113, wherein the method comprises administering the nucleic acid encoding the TDP-43 variant, wherein the nuclease agent cleaves the endogenous TARDBP genomic locus in the one or more cells, the nucleic acid encoding the TDP-43 variant is inserted at or recombines with the cleaved endogenous TARDBP genomic locus, wherein the TDP-43 variant is expressed from the endogenous TARDBP genomic locus and replaces expression of the endogenous TDP-43.

115. The method of any one of claims 102-114, wherein endogenous TDP-43 in the one or more cells is prone to aggregation, and wherein the method reduces TDP-43 aggregation in the one or more cells.

116. The method of any one of claims 102-115, wherein there is aberrant splicing regulation by endogenous TDP-43 in the one or more cells, and wherein the TDP-43 variant rescues aberrant TDP-43 splicing regulation in the one or more cells.

117. The method of any one of claims 102-116, wherein there is aberrant subcellular distribution of endogenous TDP-43 in the one or more cells, and wherein the TDP-43 variant rescues aberrant subcellular distribution of endogenous TDP-43 in the one or more cells.


SEQUENCE LISTING (73 sequences)

SEQ ID NO: 1 - protein, 414 aa, Homo sapiens
  DOMAIN 82..98: note = NLS
  DOMAIN 106..176: note = RRM1
  DOMAIN 191..262: note = RRM2
  DOMAIN 274..414: note = PLD
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - DNA, 1245 bp, Homo sapiens
  source 1..1245: mol_type = other DNA
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtag

SEQ ID NO: 3 - DNA, 4185 bp, Homo sapiens
  source 1..4185: mol_type = other DNA
attttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccaca

SEQ ID NO: 4 - protein, 298 aa, Homo sapiens
MPQMLAGEIWCMLSTIQKVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 5 - protein, 141 aa, Homo sapiens
GRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 6 - protein, 28 aa, Homo sapiens
NPAMMAAAQAALQSSWGMMGMLASQQNQ

SEQ ID NO: 7 - protein, 353 aa, Homo sapiens
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 8 - DNA, 1062 bp, Homo sapiens
  source 1..1062: mol_type = other DNA
atggagaaaactttagaaactgttcctttggagaggaaaaagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactga

SEQ ID NO: 9 - DNA, 3664 bp, Homo sapiens
  source 1..3664: mol_type = other DNA
agtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagaaaactttagaaactgttcctttggagaggaaaaagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaattgtgtaattatagactttttattttaaataagttaaggagtgggtagtataattaaggtccgttgcaaagctgttgttatatttgtataagataaatgctggtcagatgtaagtgtgttgtctgcaattcatcaggattaaattatgtagataacttaagggatatctctgcaaggagaaacacctttttagatcttttagatgctgcttcttcaatgcaaggaaaggaaataaccccagcgaggtactcttcagggacacaggtctagtacaagagaactcttgacggctactaagttcagccagtcttaaaaaactgtgctgtttctacaaaactttaactacagtagtttataaggatgccaacgaaagctgagggtgtagagcaaaatagttctaagcttcagttaaacttctttaggtaagatcttatttacttttcctttcttaattttcctccctaaaagataaactaatactcttaaatggtctttcagtatagtggttcttacgtagtttaacatagctataaattgagtttaacaatttataaactcaagagaataatttttataaaccctgttttccaatctgtcatttacttaaattattttggttgtttttccctttttttccttcttttcccaccccctccccctccatgtgaagatttgggtgcttaacatatcatttttttccctgccggaattttagcattgatatgaaccatggacaagtatattctgctgccacaaagactgtaaagtgcttcatttcaacagctgaggcaagccaagtgatcattaataaagcttttcttggttccttcagtggtgttggtagtaaaatggtaggtaaaagttaggctgcaagttcaataaatcatgagatttcccatcgttacacccttgtgtattcacatttcttggatcaaacattttgagtgaactaggggtttttattaaagacatttgttgtatttatggttgtaactgtacatgcttatcaggatgagactgaaagaaggtagggcaaaaatggttgaatctattttcagatagtagttcatacttgagtgaagtgtcttgtctgcattatgaagcctggtatgtatccagtactaaataggtgggttaaatgtggtaattctagttcagtgtcttaccctgaagagaaagttgtaggttggctgttgaaattcattccttagatatgatcagtttgattgcccggctttattgcctttacaggaatgtgatactcagggcttactctatacaccaatgagtcttctttgatcctaagaccaccactgaagttgtttaggttcttttggacaaacatgataaacttcttcagatactttttttttcctttggcaggaaggtgtcttgctgcaggtaactaatgaagaagtggtcaaccacagagtcttcaagaaataagaaattctgtaccatctgaaagtagttcttgttggtgccttcatttaaaaagcactctttaaaataaaagggaaatgttttctgataaaacaaacatttagttgaggttcttgatataaaacaattacaaaatgagtgttgtttgtaaaacagtaacatcaaattggctagagagataaatgtatcatgttttaaattaggttttgtgagtagacagattacaattctattttaaatataaagtttataaaataaatactttttgtatccaaatacttggtgtaatgtttacacataaaatgtgtgaatcttgttctataaatatttggttgtctaaaagatcaccatcccctaaatttttaaaagcagtttcacaaagctatgcatattttaatattaacaggtaaatgagaagagcattgtggacattattggctgtccccaataaaatgctgttcattatgcactgta

SEQ ID NO: 10 - protein, 341 aa, Homo sapiens
MEREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 11 - DNA, 1026 bp, Homo sapiens
  source 1..1026: mol_type = other DNA
atggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactga

SEQ ID NO: 12 - DNA, 3628 bp, Homo sapiens
  source 1..3628: mol_type = other DNA
agtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaattgtgtaattatagactttttattttaaataagttaaggagtgggtagtataattaaggtccgttgcaaagctgttgttatatttgtataagataaatgctggtcagatgtaagtgtgttgtctgcaattcatcaggattaaattatgtagataacttaagggatatctctgcaaggagaaacacctttttagatcttttagatgctgcttcttcaatgcaaggaaaggaaataaccccagcgaggtactcttcagggacacaggtctagtacaagagaactcttgacggctactaagttcagccagtcttaaaaaactgtgctgtttctacaaaactttaactacagtagtttataaggatgccaacgaaagctgagggtgtagagcaaaatagttctaagcttcagttaaacttctttaggtaagatcttatttacttttcctttcttaattttcctccctaaaagataaactaatactcttaaatggtctttcagtatagtggttcttacgtagtttaacatagctataaattgagtttaacaatttataaactcaagagaataatttttataaaccctgttttccaatctgtcatttacttaaattattttggttgtttttccctttttttccttcttttcccaccccctccccctccatgtgaagatttgggtgcttaacatatcatttttttccctgccggaattttagcattgatatgaaccatggacaagtatattctgctgccacaaagactgtaaagtgcttcatttcaacagctgaggcaagccaagtgatcattaataaagcttttcttggttccttcagtggtgttggtagtaaaatggtaggtaaaagttaggctgcaagttcaataaatcatgagatttcccatcgttacacccttgtgtattcacatttcttggatcaaacattttgagtgaactaggggtttttattaaagacatttgttgtatttatggttgtaactgtacatgcttatcaggatgagactgaaagaaggtagggcaaaaatggttgaatctattttcagatagtagttcatacttgagtgaagtgtcttgtctgcattatgaagcctggtatgtatccagtactaaataggtgggttaaatgtggtaattctagttcagtgtcttaccctgaagagaaagttgtaggttggctgttgaaattcattccttagatatgatcagtttgattgcccggctttattgcctttacaggaatgtgatactcagggcttactctatacaccaatgagtcttctttgatcctaagaccaccactgaagttgtttaggttcttttggacaaacatgataaacttcttcagatactttttttttcctttggcaggaaggtgtcttgctgcaggtaactaatgaagaagtggtcaaccacagagtcttcaagaaataagaaattctgtaccatctgaaagtagttcttgttggtgccttcatttaaaaagcactctttaaaataaaagggaaatgttttctgataaaacaaacatttagttgaggttcttgatataaaacaattacaaaatgagtgttgtttgtaaaacagtaacatcaaattggctagagagataaatgtatcatgttttaaattaggttttgtgagtagacagattacaattctattttaaatataaagtttataaaataaatactttttgtatccaaatacttggtgtaatgtttacacataaaatgtgtgaatcttgttctataaatatttggttgtctaaaagatcaccatcccctaaatttttaaaagcagtttcacaaagctatgcatattttaatattaacaggtaaatgagaagagcattgtggacattattggctgtccccaataaaatgctgttcattatgcactgta

SEQ ID NO: 13 - protein, 152 aa, Homo sapiens
GRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 14 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

SEQ ID NO: 15 - DNA, 1119 bp, Homo sapiens
  source 1..1119: mol_type = other DNA
atgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaa

SEQ ID NO: 16 - DNA, 3744 bp, Homo sapiens
  source 1..3744: mol_type = other DNA
agatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaatgaaatgacaactgtatggatttgggactggcagagatttggactttccctacccactccccctgataataatgttgaatgcttctatcacaattcaagttcaaagctctgccagggaatagaaactagctgctggctaatgccgctccataaatccgcagatttgaagtgtctgggaggccttttaaaaataactaatatcagaaagcattttaatgaacgtaaagataggcttacattaaaggaaaactgcagtttgactgggttctggttgggtgggccttcagaggttctatcagttaactatactaattaatttggagattcaaaccataccaatagaaactaaatttttctacataatttcatgttaactgcagtttccctttatggcacaaggggtcacacaacctacctaaaatgttaattgtatagaaaatgatttctaaacctttaacagttaatatccaataatgtgttatttgtacatagattcttttgagccttacctttggtgctctctcaatttcagtgctattgtacctagtagcatagagatttgtctatcaataggactgtatattccatcccatgggtgtgttggaagtttggggaggaggagggtggtgggaggtgggtgggaggtggtggtgggtgggaaagcatgggtgatagttccatgatactggctgagtttgcaatagcaggtggaaccctaactattgagggagtttgcagatacctcaggattcgtgacaatgcctttaaagatccaggagagatgttcagctactaggaactgctagcaagtatagcgcgaatggctccgagctcagacactctacagctgagagtagacacttgtggtatgtggagtacagataagccaggggcaggccacggcacgctccatgaaagctaggagggagtgaaatatcagtgatcatcgcaaggaaggaggcagacaagagtaaggcacacctgactcttaggactagcagtcagaaccaggaggaaaggttttattgctatgcgggtaggtaagaacagattttacttacatccatatagttacttaaagtccagttttctgttaaacatttttcttaatatattgagccaaaactagtccagttaagctgaacttggtttttctggagatgaattgttttaaattgacaccctattgatggctcccagttgaaggaagtgagcacattatttgtactgtgaatataaatttttgcccttttatttatcttcctttgacccatttccttaaaataatggctcaaagtaatagacttccccaaatggtggggggatgggtgggttattaatgggaggtatggggggtttagcttgagatgggacttggtcttagagctagttctaaaggttgtttacttttctagggaggagtctgctactagtcttatcagctcttaaaaacagaaactcatctgtccaagttcgtggcagaaaggtaagtttttacaaattagtgctcagcaaaagaatgccctgcgttcccaaagtaaaagaatgacaagctgtaccttaaaccaaaacacttcgtaatctcatccaattgcaaaaagagttattagccaaccaggtattcccagtagtgacagtggatataactgtgtagtcattcacctctgcttatatgaatactttacaacctcttttgccttttgcaggaacgtccttgtgaagacctttatctgagccactgtacttcgttatcactgccatgcagtttacatgagctgttctgcagctcaaattccattttgtgaatgggtttttttttttaataaactgtatttaactta

SEQ ID NO: 17 - protein, 320 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGSNFGGGGSYNDFGNYNNQSSNFGPMKGGNFGGRSSGPYGGGGQYFAKPRNQGGYGGSSSSSSYGSGRRF

SEQ ID NO: 18 - DNA, 963 bp, Homo sapiens
  source 1..963: mol_type = other DNA
atgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaa

SEQ ID NO: 19 - DNA, 3588 bp, Homo sapiens
  source 1..3588: mol_type = other DNA
agatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaatgaaatgacaactgtatggatttgggactggcagagatttggactttccctacccactccccctgataataatgttgaatgcttctatcacaattcaagttcaaagctctgccagggaatagaaactagctgctggctaatgccgctccataaatccgcagatttgaagtgtctgggaggccttttaaaaataactaatatcagaaagcattttaatgaacgtaaagataggcttacattaaaggaaaactgcagtttgactgggttctggttgggtgggccttcagaggttctatcagttaactatactaattaatttggagattcaaaccataccaatagaaactaaatttttctacataatttcatgttaactgcagtttccctttatggcacaaggggtcacacaacctacctaaaatgttaattgtatagaaaatgatttctaaacctttaacagttaatatccaataatgtgttatttgtacatagattcttttgagccttacctttggtgctctctcaatttcagtgctattgtacctagtagcatagagatttgtctatcaataggactgtatattccatcccatgggtgtgttggaagtttggggaggaggagggtggtgggaggtgggtgggaggtggtggtgggtgggaaagcatgggtgatagttccatgatactggctgagtttgcaatagcaggtggaaccctaactattgagggagtttgcagatacctcaggattcgtgacaatgcctttaaagatccaggagagatgttcagctactaggaactgctagcaagtatagcgcgaatggctccgagctcagacactctacagctgagagtagacacttgtggtatgtggagtacagataagccaggggcaggccacggcacgctccatgaaagctaggagggagtgaaatatcagtgatcatcgcaaggaaggaggcagacaagagtaaggcacacctgactcttaggactagcagtcagaaccaggaggaaaggttttattgctatgcgggtaggtaagaacagattttacttacatccatatagttacttaaagtccagttttctgttaaacatttttcttaatatattgagccaaaactagtccagttaagctgaacttggtttttctggagatgaattgttttaaattgacaccctattgatggctcccagttgaaggaagtgagcacattatttgtactgtgaatataaatttttgcccttttatttatcttcctttgacccatttccttaaaataatggctcaaagtaatagacttccccaaatggtggggggatgggtgggttattaatgggaggtatggggggtttagcttgagatgggacttggtcttagagctagttctaaaggttgtttacttttctagggaggagtctgctactagtcttatcagctcttaaaaacagaaactcatctgtccaagttcgtggcagaaaggtaagtttttacaaattagtgctcagcaaaagaatgccctgcgttcccaaagtaaaagaatgacaagctgtaccttaaaccaaaacacttcgtaatctcatccaattgcaaaaagagttattagccaaccaggtattcccagtagtgacagtggatataactgtgtagtcattcacctctgcttatatgaatactttacaacctcttttgccttttgcaggaacgtccttgtgaagacctttatctgagccactgtacttcgttatcactgccatgcagtttacatgagctgttctgcagctcaaattccattttgtgaatgggtttttttttttaataaactgtatttaactta

SEQ ID NO: 20 - protein, 267 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGSYNDFGNYNNQSSNFGPMKGGNFGGRSSGPYGGGGQYFAKPRNQGGYGGSSSSSSYGSGRRF

SEQ ID NO: 21 - protein, 123 aa, Homo sapiens
GRSGSGNFGGGRGGGFGGNDNFGRGGNFSGRGGFGGSRGGGGYGGSGDGYNGFGNDGSNFGGGGSYNDFGNYNNQSSNFGPMKGGNFGGRSSGPYGGGGQYFAKPRNQGGYGGSSSSSSYGSG

SEQ ID NO: 22 - protein, 141 aa, synthetic construct
GRFGGNPGGFGNQGGFGNSRGGGAGFGNNQGSFMGGGMNFGAFSINPAMYAAAQFALQSSFGMMGMLYSQQNYSGPSGFNQNQGNYQREPNQAFGSGNYSYSGSYSGAAIGFGSASNAGFGSGFNGGFGSSMDSKSSGFGM

SEQ ID NO: 23 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGFGNNQGSFMGGGMNFGAFSINPAMYAAAQFALQSSFGMMGMLYSQQNYSGPSGFNQNQGNYQREPNQAFGSGNYSYSGSYSGAAIGFGSASNAGFGSGFNGGFGSSMDSKSSGFGM

SEQ ID NO: 24 - protein, 28 aa, synthetic construct
YGGGPGGGNFGGSPGYGGGRGGYGGGGP

SEQ ID NO: 25 - protein, 141 aa, synthetic construct
GRFGGNPGGFGNQGGFGNSRGGGAGLGNNQGSNMGGGMNFGAFSIYGGGPGGGNFGGSPGYGGGRGGYGGGGPSGPSGNNQNQGNMQREPNQAFGSGNNSYSGSNSGAAIGWGSASNAGSGSGFNGGFGSSMDSKSSGWGM

SEQ ID NO: 26 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSIYGGGPGGGNFGGSPGYGGGRGGYGGGGPSGPSGNNQNQGNMQREPNQAFGSGNNSYSGSNSGAAIGWGSASNAGSGSGFNGGFGSSMDSKSSGWGM

SEQ ID NO: 27 - protein, 148 aa, synthetic construct
GRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGG

SEQ ID NO: 28 - protein, 421 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGG

SEQ ID NO: 29 - DNA, 22 bp, synthetic construct
  source 1..22: mol_type = other DNA
gcatgagttagagttctgtctg

SEQ ID NO: 30 - DNA, 21 bp, synthetic construct
  source 1..21: mol_type = other DNA
cttccgccacagacatatttc

SEQ ID NO: 31 - DNA, 21 bp, synthetic construct
  source 1..21: mol_type = other DNA
ctacttcgtactctccagctg

SEQ ID NO: 32 - DNA, 21 bp, synthetic construct
  source 1..21: mol_type = other DNA
gatgctggctgtatgacgatc

SEQ ID NO: 33 - DNA, 21 bp, synthetic construct
  source 1..21: mol_type = other DNA
gagaagatcagcttgaagagg

SEQ ID NO: 34 - DNA, 21 bp, synthetic construct
  source 1..21: mol_type = other DNA
caccacaatgtcatcatcttc

SEQ ID NO: 35 - DNA, 22 bp, synthetic construct
  source 1..22: mol_type = other DNA
gcagaatcttgacaacatcagg

SEQ ID NO: 36 - DNA, 21 bp, synthetic construct
  source 1..21: mol_type = other DNA
gctttctttccagaaggttcc

SEQ ID NO: 37 - DNA, 19 bp, synthetic construct
  source 1..19: mol_type = other DNA
caccgaagaagctggaatc

SEQ ID NO: 38 - DNA, 19 bp, synthetic construct
  source 1..19: mol_type = other DNA
ccaggtgatatcggtgatg

SEQ ID NO: 39 - DNA, 22 bp, synthetic construct
  source 1..22: mol_type = other DNA
gtttcatgagcattggcggttc

SEQ ID NO: 40 - DNA, 22 bp, synthetic construct
  source 1..22: mol_type = other DNA
gtagtctccagcagtgacactg

SEQ ID NO: 41 - DNA, 21 bp, synthetic construct
  source 1..21: mol_type = other DNA
accaccatggagaaggccggg

SEQ ID NO: 42 - DNA, 21 bp, synthetic construct
  source 1..21: mol_type = other DNA
cagtgatggcatggactgtgg

SEQ ID NO: 43 - protein, 414 aa, Mus musculus
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 44 - DNA, 1245 bp, Mus musculus
  source 1..1245: mol_type = other DNA
atgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcattaacccagcgatgatggctgcggctcaggcagcgttgcagagcagttggggtatgatgggcatgttagccagccagcagaaccagtcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtag

SEQ ID NO: 45 - DNA, 7454 bp, Mus musculus
  source 1..7454: mol_type = other DNA
ctcggaaggccgagtggccgttctgtccttcatctgtcagtttttcagacccagctgttttcattgttgcgtttctttacttttttctatacgccgaagagcctgctagcatccgagcctctgggaggagagagcgcctgtggcttccctcggagagcgcccctcctgcagggaagccagtgggagaggccgaaggcgggcgagggcgggaggcggccctagcgccattttgtgggcacggagcggtagcgcggctgttgtcggattccttcccgtctgtgcttcctccttgtgcttcctagcagtggcctagcggagatttaagcaaagatgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcattaacccagcgatgatggctgcggctcaggcagcgttgcagagcagttggggtatgatgggcatgttagccagccagcagaaccagtcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtaggtggtggggggtggttagtaggttggttattaggttaggtagatttagaatggtgggattcaaatttttctaaactcatggtaagtatattgtaaaatacatatgtactaaaattttcagattggtttgttcagtgtggagtatattcagcagtatttttgacatttttctttagaaaaaaagaggggaaagctaaatgaattttataagttttgttatataaagggttaaaatactgagtgggtgaaagtgaactgctgtttgcctaattggtaaaccaacactacaattgatctcagaaggtttctctgtaatattctatcattgaaattgttaatgaattctttgcatgttcagagtagaaaccattggttagaactacattcttttctccttattttaatttgaatcccaccctatgaattttttccttaggaaaatctccatttgggagatcatgatgtcatggtgtttgattcttttggttttgtttttaacacttgtcttccttcatatacgaaagtacaatatgaagccttcatttaatctctgcagttcatctcatttcaaatgtttatggaagaagcacttcattgaaagtagtgctgtaaatattctgccataggaatacttctgtctacatgctttctcatccaagaattcgtcatcacgctgcacaggctgcgtctttgacggtgggtgttccatttttatccgctactctttatttcatggagtcgtatcaacgctatgaacgcaaggctgtgatatggaaccagaaggctgtttgaacttttgaaaccttgtgtgggattgatggtggtgccgaggcatgaaaggctagtatgagcgagaaaaggagagcgcgtgcagagacttggtggtggaaaatggatattttttaacttggagagatgtgtcactcaatcctgtggctttggtgagagagtgtgcagagagcaatgatagcaaataacgtacgaatgttttacatcaaaggacatccacatcagttggaagactttgagttttgttcttaggaaacccactttagttgaatgtgttaagtgaaatacttgtacttccctccccctctgtcaactgctgtgaatgctgtatggtgtgtgttctcctctgttactgatctggaagtgtgggaacgtgaactgaagctgatgggctgcgaacatggactgagcttgtggtgtgctttgcaggagaacttggaagcagagttcaccagtgagctcaggtgtctcaaagaagggtggaagttctcatgtctgttagctattcataagaatgctgtttgctgcagttctgtgtcctgtgcttggatgcttttttataagagttgtcattgttggaaattcttaaataaaactgatttaaataatatgtgtctttgttttgcagccctgaatgcaaagaattcatagcagttaattcccctttttgacccttttgagatggaactttcataaagtttcttggcagtagtttattttgcttcaaataaacttatttgaaaagttgtctcaagtcaaatggattcatcacctgtcatgcattgacacctgatacccagacttaattgctatttgttcttgcattgtccaaagtgaaagtttttctttggtttgtttttaatttagtttttcttaagtctgggtgaccgcacctaaaatggtaagcagttaccctctggcttgttctgagtgcctctgtgcatttgattttctatttacatgctgtataaatctccactggggaatcatgccttctaaaaatatttgggagagggcaaaagagttgatttctaatgctttgtagcagagcatatcaatgggaaagaaggttaagcacctttctgtttgggatttgaaaagtggaattaattgcaatagggatgaagtagaagaaaccaagaaaccatgtgcctgaaatacattaagaagcctgattgatagctttaagaactagtagggtgggttgtcttacctgtggcagtcttaagtgaggtaggcttttgccctcctgaatgtgggggttatgtagtgatgaatatgctcacaaaatcagattagactgtcaatgcattgttaatgtaaaagcaataatacattgattattgtacttttcctgtaactactgagaccggaggcgctccttttctaactggaagaatgggacagtttttgtgttggtagtttttcctaatgcccttacctaaatagattatgataaataggtttgtcattttgcaagttgcgtgttttaaaattttatatccgttagagacttgttatgaacacattgtttcattatacagtatcctctgtaaaaggatcgtgagttattgtaagtttttttctctgcatctaaccctgcatgatttccaaaccctgtgcatctgaattttgcattttagcactgtttgcactgttactcagcagcagtaacatggtaacattaaaatggttttcggggacctccaaagacggccaggagtcctggggtaagttacttgtcaatggcatggttttgatcccttttttacacttgttaaagacttactggtcatagaagtctttcagtgttgatcagccttttaacatgtttatggatgacatagctgtagttagttacttgccgtaaatgaggttttagaaataaactacttggcaaagatttggttttgaaagtctggtcatcaaaacgcgttcattccttagaaataatgaagaacaactctttgaaccacagttgaataaaaggttttcttgccaccaacagtttagtgtctggagtcttactggaagaaaaaaaaattctatatcatgacaatgctagaaaagttaaggtgacttatgtgggaagatgcaatatagcattttcatcctttaaaatttgagtctccaggtgggtgtggtggcccatgcttttaatcccagaattggtgtaaatgagttgtaggccagctatttccccatcttgaggcaccctgtcttgccttgttggaagagccagttaaaatcaaacatgacctttaaggtcagcatcttagcagaagagcagtttatttcaggataacttactgtttttgatacataagcaaatgactgtaccttgtacagttacggttgacttccctgagcccaacgctcacctaagaaaagtgggctgggtatagtgaaacacctgttaaggtttttggaatgatttgctaaattgcccttgtaaagggtaaaatgctgtttcgtgttctttttatctgacaatttggtgaatctggtagaacatgcctatatcccaatattctggaatggacttggtgttaatttaatagctgatctaatgtgaaggtcacacacctgctttccgcttaccttccaaaaggtattctggaaccactcagaagttactcagaaagtaagagcactttctgagctcattaagaccaaatctacacactagacacaacaagccctttgttggccagaaatggaaacagccagtataaaataagtagattgtagggaatcaaatacaacttgttttttctgtgttgggggttggccaagcactgttaaacacaaatcaaagctgatattggcaagtgtttggacctgtaacaatctcacctgctctgattttggggtaggctgtcattcttaggtttgttactaagcttcccaggtacttggcgatatgaggaacaatgattggacgatgcaaattagaaattacttattatgttctcaatccagggaatatagttgatgacttttgtgtagaccccatactggtctgccgccccacaattaatggaaccccaggaaactattcctcccacaaaccatcgctgtgtctcattatctagaaacactaatgcccccccactgtcacctctgcagctgtccttgccaccagtctctaagccagcacagagcatgttagcgcttactcttactcctggatagagcttttcatacacggcggtacatttttggtggtcagcaattggtatgtccacaaacattaggtttctagcaagaagccccttctgggttaacccccagccagccacagttccagtgaagtctgttctcattaaggatgcagcttcttttcgcggtaggcaaacaggcatgatgcttccgttgattgtgactttgttcttgagtttaatcaatgctatatcattgtcaaaaccagcaccgtgagtgtagccttcatgtataaagatttcctcgggccaggcttgagtgtaatgaggtgagagccttttgaggatgcccattcggatgttcagggaggacgctgccattcttttctcatatacagcatgagcggctgttaggacccaattgtcatgtataagtgcacctgctgctgctgtagtttgacccagcaacaagacttgccaaggaaagtcaccaggctttgcaggctgccctccaactatgcgtcctcctatagtgtgtgtggacagcccacaaactgagggataaaaaacaagtatttaatgccctacaaattgataggcatcctccctgttgagtgaggctatttaagtttttgtttagcctgctcacctccttgtaaagatcagtaagagatctcagagattgttttgctgaaagagaacagcatgagggagtctggacaggacttgtgtgcaggaggacatgacactacttagaggccaaagacaaccccctcaccacaccctgagctgcttgtttttctctttttgggctctctgggaattctgggaaagcaggaactatgatttacaaagtttctgttgtctttaaatgtaagactctaaattacaatgttgcagcaatagccaaagtgcttctggttcaaaaattggtaattttggtctggtggagccctccaaacacttttaccgttctttgcaaactgagggctcaggaatgcaacacatgttctttattgtggttgtgcactttgattaaaacttggaagccgcatgtcagccaaatacaaggctagaaaactaatttaaaccagctaacacgggggtaatgagtgtattattacctttcaattaaaaaaaaagcactctcaactgttgttggagccaattctggtaaagaaatttaagtactattaaaaggcaaattgcattaatgtttaaaaatcttgatgtcgttgaaaacaattgcttagggaaataatgaagttattagctttggggtttaatagcatttttacagagaagaaaagtaacaagagttcttggttataaatgtataaacggtttgagataatttaagaaatcatttaattttttatgcttgcctagttataaggtcaaaaacaatcaagtgcatgatgcacctagcttccgtgtggaaggggaaatgtgagcacactgttgggaaacactaagctccagcctcagccaagtgctgagctttctgcctccccagccagaccctgcctattgtctgccagctactctgtcagctatgaatctcttttataaatggcgtccattaccaggctcacaaaccggggggagtttttctcctttggagctcgtccagaatccatcagcctcacacacatatttacctgcaagtcattggaaaagcaaaaatgtttagctgtagttgtcatttgcttgaataaccccttgaaaaatgttgattcttgagcatctgtggtggggagaggtgtgtgaataaccattttacatgatttcataaataggtgtctgcattaccatgtttgcttgcaaagtggaaaccttttagatgtgtaacttgaatatgtatcaagatctcaagtgcttaatgataaggttttgacttgttaaattaaaccatttggaatatattgtg

SEQ ID NO: 46 - protein, 141 aa, Mus musculus
GRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 47 - protein, 28 aa, Mus musculus
NPAMMAAAQAALQSSWGMMGMLASQQNQ

SEQ ID NO: 48 - protein, 353 aa, Mus musculus
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGSYNDFGNYNQQPSNYGPMKSGNFGGSRNMGGPYGGGNYGPGGSGGSGGYGGRSRY

SEQ ID NO: 49 - DNA, 1062 bp, Mus musculus
  source 1..1062: mol_type = other DNA
atggagaaaactttagaaactgttcctttggagaggaaaaagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaa

SEQ ID NO: 50 - DNA, 2403 bp, Mus musculus
  source 1..2403: mol_type = other DNA
aagcggcggtctcggctccagcggcagtagcagcggcgccggtcccgtgtgcaggagctcctttgcggcccagtttcttggccatcgcctgctctccccacagcgccaggacgagtcccgtgcgcgtccgtccgcggaggtctttctcatctcgctcggctgcgggaaatcgggctgaagcgactgagtccgcgatggagaaaactttagaaactgttcctttggagaggaaaaagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaagcttcttcctacttaccatggcattgatatgaaccatggacaagtatattctgctgccacaaagactgtaaagtgcttcatttcaacagctgaggcaagccaagtgatcattaataaagcttttcttgcttccttcagtggtgttggtagtaaaatggtaggtaaaagttaggctgcaagttcaataaatgagatttacctatcattccacccttgtgtattcattcacctatcctggttcaagcagtttgagtcaactaggcatttaaaggcattgtgtttattgtggtaacaactatacatgcttatcagggatgagtctgacagtgttttcttacagtagaagtttctgtgcaacagcagcagcaaaaccctctatgtaccctgtactaaatagttggggtaaaatgtggtaattttttgcccattgtcctgccctaaaatgttgtaggttggctattgaaatttcctgaaacagccctcttgatttctaggctttattgccttttcagaaatgtgatattgagcgcttattatacacatcagtgagtctgctttaatcctaaacccaccactgaagttacttaggttctgtggacaaacatgataaacttcttcagatactttttttttcctttggcaggaaggtgtcttgctgcaggtaactaatgaagaagtggtcaaccacagagtcttcaagaaataagaaattctgtaccatctgaaagtagttcttgttggtgccttcatttaagaagcacttttaagaaaagggaatgttttctgatttaaaacttcatttagtacaggttcttaataaaattacaaaaatgctgattgtaaagggtaacatcaaattagcaaaggataatgtattgttttaggatttgtgagtagacagataagagctctattttaaatacaagttttataaaataaatacttttgtatccaaattacttggtgtaatgcttacacataagtgtgaatcttgttctatcaacatcagttgtctacaagatcgacactataaatgctagactagttttgcacagaactatacatttagttctcattcaatactaataggagaaaaaagcattaataggcattatttactgtctaataaaatataccttactatgaa

SEQ ID NO: 51 - DNA, 2252 bp, Mus musculus
  source 1..2252: mol_type = other DNA
agtagcagcggcgccggtcccgtgtgcaggagctcctttgcggcccagtttcttggccatcgcctgctctccccacagcgccaggacgagtcccgtgcgcgtccgtccgcggaggtctttctcatctcgctcggctgcgggaaatcgggctgaagcgactgagtccgcgatggagaaaactttagaaactgttcctttggagaggaaaaagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaagcttcttcctacttaccatgggaaggtgtcttgctgcaggtaactaatgaagaagtggtcaaccacagagtcttcaagaaataagaaattctgtaccatctgaaagtagttcttgttggtgccttcatttaagaagcacttttaagaaaagggaatgttttctgatttaaaacttcatttagtacaggttcttaataaaattacaaaaatgctgattgtaaagggtaacatcaaattagcaaaggataatgtattgttttaggatttgtgagtagacagataagagctctattttaaatacaagttttataaaataaatacttttgtatccaaattacttggtgtaatgcttacacataagtgtgaatcttgttctatcaacatcagttgtctacaagatcgacactataaatgctagactagttttgcacagaactatacatttagttctcattcaatactaataggagaaaaaagcattaataggcattatttactgtctaataaaatataccttactatgaattgtgcactgctacaacttgatgctgaacactaatggtggcattttgaaaccatggattttaaccagatagtgttttgagttaccacttttgcagaacaccaaaacactgatttttattccaattgtgaatgggtagcagtggttaaggtactgtggtttaagagtaatgggaaattgatttaaaacaaccagagttaacttcatttgcttacgccaataaagcaatatatacaggtaagtcaatggctaaaaaatgtgagctcttaacatcttgtaaagtgtatcaacagtcattctaaatacttggcttttggctagaaaagctagcaaacccattaggtagtgttgtgcatgccctcagtcccagcacttgggaagttaaaggctatgttccagacaaggctgcatgtgagtctcatacacatacccgtcgccattaagagtttgtttttggacttcaaagcaaaataaagtctcacacaattgttttttaaa

SEQ ID NO: 52 - protein, 341 aa, Mus musculus
MEREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGSYNDFGNYNQQPSNYGPMKSGNFGGSRNMGGPYGGGNYGPGGSGGSGGYGGRSRY

SEQ ID NO: 53 - DNA, 1026 bp, Mus musculus
  source 1..1026: mol_type = other DNA
atggagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaa

SEQ ID NO: 54 - DNA, 2265 bp, Mus musculus
  source 1..2265: mol_type = other DNA
gttagtggggggaaggttctagaaaagcggcggtctcggctccagcggcagtagcagcggcgccggtcccgtgtgcaggagctcctttgcggcccagtttcttggccatcgcctgctctccccacagcgccaggacgagtcccgtgcgcgtccgtccgcggaggtctttctcatctcgctcggctgcgggaaatcgggctgaagcgactgagtccgcgatggagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaagcttcttcctacttaccatgggaaggtgtcttgctgcaggtaactaatgaagaagtggtcaaccacagagtcttcaagaaataagaaattctgtaccatctgaaagtagttcttgttggtgccttcatttaagaagcacttttaagaaaagggaatgttttctgatttaaaacttcatttagtacaggttcttaataaaattacaaaaatgctgattgtaaagggtaacatcaaattagcaaaggataatgtattgttttaggatttgtgagtagacagataagagctctattttaaatacaagttttataaaataaatacttttgtatccaaattacttggtgtaatgcttacacataagtgtgaatcttgttctatcaacatcagttgtctacaagatcgacactataaatgctagactagttttgcacagaactatacatttagttctcattcaatactaataggagaaaaaagcattaataggcattatttactgtctaataaaatataccttactatgaattgtgcactgctacaacttgatgctgaacactaatggtggcattttgaaaccatggattttaaccagatagtgttttgagttaccacttttgcagaacaccaaaacactgatttttattccaattgtgaatgggtagcagtggttaaggtactgtggtttaagagtaatgggaaattgatttaaaacaaccagagttaacttcatttgcttacgccaataaagcaatatatacaggtaagtcaatggctaaaaaatgtgagctcttaacatcttgtaaagtgtatcaacagtcattctaaatacttggcttttggctagaaaagctagcaaacccattaggtagtgttgtgcatgccctcagtcccagcacttgggaagttaaaggctatgttccagacaaggctgcatgtgagtctcatacacatacccgtcgccattaagagtttgtttttggacttcaaagcaaaataaagtctcacacaattgttttttaaa

SEQ ID NO: 55 - protein, 301 aa, Mus musculus
MEREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGNYGSGSYNDFGNYNQQPSNYGPMKSGNFGGSRNMGGPYGGGNYGPGGSGGSGGYGGRSRY

SEQ ID NO: 56 - DNA, 906 bp, Mus musculus
  source 1..906: mol_type = other DNA
atggagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaa

SEQ ID NO: 57 - DNA, 2145 bp, Mus musculus
  source 1..2145: mol_type = other DNA
gttagtggggggaaggttctagaaaagcggcggtctcggctccagcggcagtagcagcggcgccggtcccgtgtgcaggagctcctttgcggcccagtttcttggccatcgcctgctctccccacagcgccaggacgagtcccgtgcgcgtccgtccgcggaggtctttctcatctcgctcggctgcgggaaatcgggctgaagcgactgagtccgcgatggagagagaaaaggaacagttccgaaagctctttattggtggcttaagctttgaaaccacagaagaaagtttgagaaactactatgagcaatggggaaagctcacagactgtgtggttatgcgggatcctgcaagcaaaagatcaagaggatttggctttgtaactttctcatccatggccgaggttgacgctgccatggctgcaaggccccattccattgatggcagggtagttgagccaaaacgtgctgtagcaagagaggagtctggaaaaccaggagcccatgtgactgtgaagaagctgtttgttggtggaattaaggaagatactgaggaacaccaccttagagattactttgaagagtatggaaaaattgatactattgaaataattaccgataggcagtctggaaagaaaagaggctttggctttgttacttttgatgaccatgatcctgtggataaaattgtcttgcaaaaatatcacaccataaatggtcacaatgcagaagttagaaaggcattgtctagacaagaaatgcaggaagtccaaagttctaggagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggaaggagcagatattaagcttcttcctacttaccatgggaaggtgtcttgctgcaggtaactaatgaagaagtggtcaaccacagagtcttcaagaaataagaaattctgtaccatctgaaagtagttcttgttggtgccttcatttaagaagcacttttaagaaaagggaatgttttctgatttaaaacttcatttagtacaggttcttaataaaattacaaaaatgctgattgtaaagggtaacatcaaattagcaaaggataatgtattgttttaggatttgtgagtagacagataagagctctattttaaatacaagttttataaaataaatacttttgtatccaaattacttggtgtaatgcttacacataagtgtgaatcttgttctatcaacatcagttgtctacaagatcgacactataaatgctagactagttttgcacagaactatacatttagttctcattcaatactaataggagaaaaaagcattaataggcattatttactgtctaataaaatataccttactatgaattgtgcactgctacaacttgatgctgaacactaatggtggcattttgaaaccatggattttaaccagatagtgttttgagttaccacttttgcagaacaccaaaacactgatttttattccaattgtgaatgggtagcagtggttaaggtactgtggtttaagagtaatgggaaattgatttaaaacaaccagagttaacttcatttgcttacgccaataaagcaatatatacaggtaagtcaatggctaaaaaatgtgagctcttaacatcttgtaaagtgtatcaacagtcattctaaatacttggcttttggctagaaaagctagcaaacccattaggtagtgttgtgcatgccctcagtcccagcacttgggaagttaaaggctatgttccagacaaggctgcatgtgagtctcatacacatacccgtcgccattaagagtttgtttttggacttcaaagcaaaataaagtctcacacaattgttttttaaa

SEQ ID NO: 58 - protein, 152 aa, Mus musculus
GRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGSYNDFGNYNQQPSNYGPMKSGNFGGSRNMGGPYGGGNYGPGGSGGSGGYGGRSRY

SEQ ID NO: 59 - protein, 320 aa, Mus musculus; identical to SEQ ID NO: 17

SEQ ID NO: 60 - DNA, 963 bp, Mus musculus
  source 1..963: mol_type = other DNA
atgtctaagtccgagtctcccaaggagccagaacagctgcggaagctcttcatcggagggctgagcttcgaaacaaccgacgagagtctgaggagccattttgagcaatggggaacactaacagactgtgtggtaatgagagatccaaacaccaagagatccaggggctttgggtttgtcacatatgccactgtggaagaagtggatgctgccatgaatgcaagaccacacaaggtggatggaagagttgtggaacctaagagagctgtctcaagagaagattctcagcgaccaggtgcccacttaactgtgaaaaagatctttgttggtggtattaaagaagacactgaagaacatcacctacgagattattttgagcagtatgggaagattgaagtgatagaaattatgactgacagaggcagtgggaaaaagaggggctttgcttttgttacctttgatgaccatgactctgtggataagattgttattcagaaataccatactgtgaatggccacaactgtgaagtaagaaaggctctgtcgaagcaagagatggctagtgcttcatccagtcagagaggtcgcagtggttctggaaactttggtggtggtcgtggaggcggttttggtggcaatgacaattttggtcgaggagggaacttcagtggtcgtggtggctttggtggcagccgtggtggtggtggatatggtggcagtggggatggctataatggatttggcaatgatggaagcaattttggaggtggtggaagctacaatgattttggcaattacaacaatcagtcttccaattttgggccgatgaagggaggaaactttggaggcaggagctctggcccttatggtggtggaggccagtactttgctaaaccacggaaccaaggtggctatggcggttccagcagcagcagtagctatggcagtggcaggaggttctaa

SEQ ID NO: 61 - DNA, 1816 bp, Mus musculus
  source 1..1816: mol_type = other DNA
ggcgggataaaagggcgagcagaaggtaggctggcgggcacgttcgttatcgtattcctttctgctctttgacgctgccgaggaagcatcgctgaaggctctcgtaactctaccgtcatgtctaagtccgagtctcccaaggagccagaacagctgcggaagctcttcatcggagggctgagcttcgaaacaaccgacgagagtctgaggagccattttgagcaatggggaacactaacagactgtgtggtaatgagagatccaaacaccaagagatccaggggctttgggtttgtcacatatgccactgtggaagaagtggatgctgccatgaatgcaagaccacacaaggtggatggaagagttgtggaacctaagagagctgtctcaagagaagattctcagcgaccaggtgcccacttaactgtgaaaaagatctttgttggtggtattaaagaagacactgaagaacatcacctacgagattattttgagcagtatgggaagattgaagtgatagaaattatgactgacagaggcagtgggaaaaagaggggctttgcttttgttacctttgatgaccatgactctgtggataagattgttattcagaaataccatactgtgaatggccacaactgtgaagtaagaaaggctctgtcgaagcaagagatggctagtgcttcatccagtcagagaggtcgcagtggttctggaaactttggtggtggtcgtggaggcggttttggtggcaatgacaattttggtcgaggagggaacttcagtggtcgtggtggctttggtggcagccgtggtggtggtggatatggtggcagtggggatggctataatggatttggcaatgatggaagcaattttggaggtggtggaagctacaatgattttggcaattacaacaatcagtcttccaattttgggccgatgaagggaggaaactttggaggcaggagctctggcccttatggtggtggaggccagtactttgctaaaccacggaaccaaggtggctatggcggttccagcagcagcagtagctatggcagtggcaggaggttctaattacatacagccaggaaacaaagcttagcaggagaggagagccagagaagtgacagggaagctacaggttacaacagatttgtgaactcagccaagcacagtggtggcagggcctagctgctacaaagaagacatgttttagacaatactcatgtgtgtgggcaaaaactccaggactgtatttgtgactaattgtataacaggttattttagtttctgttctgtggaaagtgtaaagcattccaacaaaaggttttactgtagacctttttcacccatgctgttgattgctaaatgtaatagtctgatcatgacgctgaataaatgtgtctttttttttttttttttaaatgtgctgtgtaaagttagtctattctgaagccatcttggtaaacttccccaacagtgtgaagttagaattccttcagggtggtgccaagttccatttggaatttatttatggttgcttgggtggagaagccattgtcttcaaaaaccttgatgtcgttaaactgccagttactattgtaacttttaatgagtttcaccattgaaagggtcatccaagcaaggtcacaatttggttataaaatggttgttggcacaccctatgcaatatcaaaattgaataacggtatcagataaaataacagatgggaatgaagcttatgtatccattatcatgtgtactcaataaacgatttaattctcttgaatggaaaaaaaaaaaaaaaaa

SEQ ID NO: 62 - protein, 268 aa, Mus musculus
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGSSSSSSYGSGRRF

SEQ ID NO: 63 - protein, 141 aa, synthetic construct
GRFGGNPGGFGNQGGFGNSRGGGAGFGNNQGGFMGGGMNFGAFSINPAMYAAAQFALQSSFGMMGMLYSQQNYSGPSGFNQSQGSYQREPNQAFGSGNYSYSGSYSGAPLGFGSASNAGFGSGFNGGFGSSMDSKSSGFGM

SEQ ID NO: 64 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGFGNNQGGFMGGGMNFGAFSINPAMYAAAQFALQSSFGMMGMLYSQQNYSGPSGFNQSQGSYQREPNQAFGSGNYSYSGSYSGAPLGFGSASNAGFGSGFNGGFGSSMDSKSSGFGM

SEQ ID NO: 65 - protein, 28 aa, synthetic construct
YGGGPGGGNFGGSPGYGGGRGGYGGGGP

SEQ ID NO: 66 - protein, 141 aa, synthetic construct
GRFGGNPGGFGNQGGFGNSRGGGAGLGNNQGGNMGGGMNFGAFSIYGGGPGGGNFGGSPGYGGGRGGYGGGGPSGPSGNNQSQGSMQREPNQAFGSGNNSYSGSNSGAPLGWGSASNAGSGSGFNGGFGSSMDSKSSGWGM

SEQ ID NO: 67 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSIYGGGPGGGNFGGSPGYGGGRGGYGGGGPSGPSGNNQSQGSMQREPNQAFGSGNNSYSGSNSGAPLGWGSASNAGSGSGFNGGFGSSMDSKSSGWGM

SEQ ID NO: 68 - protein, 148 aa, synthetic construct
GRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGSYNDFGNYNQQPSNYGPMKSGNFGGSRNMGGPYGGGNYGPGGSGGSGGYGG

SEQ ID NO: 69 - protein, 421 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGSYNDFGNYNQQPSNYGPMKSGNFGGSRNMGGPYGGGNYGPGGSGGSGGYGG

SEQ ID NO: 70 - DNA, 1266 bp, synthetic construct
  source 1..1266: mol_type = other DNA
atgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagaggaggaaactttggttttggggattctcgtggtggcggtggcaattttggaccaggaccaggaagcaactttagggggggatctgatggatacggaagtggacgtggatttggggatggctataatgggtatggaggaggacctggaggtggcaattttggaggtagccctggttatggaggaggaagaggaggatatggtggtggaggacctggatatggcaaccagggtgggggctacggaggtggttatgacaactatggaggaggaaattatggaagtggaagttacaatgattttggaaattataaccagcagccttctaactatggtccaatgaagagtggaaactttgggggtagcaggaacatgggaggaccatatggtggagggaactatggtcctggaggaagtggaggaagtggtggctatggtggatag

SEQ ID NO: 71 - DNA, 1245 bp, synthetic construct
  source 1..1245: mol_type = other DNA
atgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcatttatggaggaggacctggaggtggcaattttggaggtagccctggttatggaggaggaagaggaggatatggtggtggaggaccttcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtag

SEQ ID NO: 72 - DNA, 1245 bp, synthetic construct
  source 1..1245: mol_type = other DNA
atgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttcggaaataaccagggtggttttatgggtggagggatgaactttggtgcttttagcattaacccagcgatgtacgctgcggctcagttcgcgttgcagagcagttttggtatgatgggcatgttatacagccagcagaactactcgggcccatctgggtttaaccaaagccagggcagctaccagagggaaccaaatcaggcttttggttctggaaattattcctacagtggttcttattctggtgccccccttggtttcgggtcagcatcaaatgcaggattcggcagtggttttaatgggggctttggctcgagcatggattctaagtcttctggcttcggaatgtag

SEQ ID NO: 73 - protein, 123 aa, Mus musculus; identical to SEQ ID NO: 21